(19) United States
(12) Patent Application Publication
(10) Pub. No.: US 2013/0149757 A1
(43) Pub. Date: Jun. 13, 2013

(54) Method for Producing Butanol and Isopropanol

(71) Applicant: Optinol, Inc., San Francisco, CA (US)

(72) Inventors: Donal F. Day, Baton Rouge, LA (US); Adam K. Hoogewind, Baton Rouge, LA (US); Sarabjit S. Randhava, Evanston, IL (US); Jack Oswald, San Francisco, CA (US); Lee Madsen, Danville, VA (US); Misook Kim, Yongin-si (KR)

(73) Assignee: OPTINOL, INC., San Francisco, CA (US)

(21) Appl. No.: 13/708,899

(22) Filed: Dec. 7, 2012

Related U.S. Application Data
(60) Provisional application No. 61/569,138, filed on Dec. 9, 2011.

(51) Int. Cl. C12P 7/16; C12N 1/20

(52) U.S. Cl. CPC C12P 7/16; C12N 1/20
USPC 435/160; 435/157; 435/252.7

(57) ABSTRACT

A novel Clostridium has been found that produces primarily n-butanol and isopropanol. Increased butanol was obtained by growing it continuously in an immobilized structure and extracting fermentation products immediately thereafter in a continuous flow extraction medium. Increased production was also achieved by fermentation in the presence of an extraction medium (such as corn oil) to decrease product inhibition followed by product separation from the fermentation broth and the extraction medium.

### CROSS-REFERENCE TO RELATED APPLICATIONS

The present application claims the benefit of U.S. Provisional Patent Application Ser. No. 61/569,138, filed Dec. 9, 2011 and entitled “Method for Producing Butanol and Isopropanol, which is incorporated by reference herein.

### BACKGROUND

Acetone butanol ethanol (ABE) fermentation by Clostridium acetobutylicum is one of the oldest known industrial fermentations. It was once second only to ethanol fermentation by yeast in its scale of production, and is one of the largest biotechnological processes ever known. Prior to the 1950s the industrial solvents acetone, n-butanol and isopropanol were produced by fermentation. The push for renewable fuels has re-ignited interest in butanol production by fermentation. Butanol is both an important industrial solvent and is widely recognized as a better fuel than ethanol. Butanol has several advantages over ethanol for fuel. While it can be made from the same feedstocks as ethanol, unlike ethanol it is compatible with gasoline and diesel at any ratio. Butanol can also be used alone as a pure fuel in existing cars without modifications; it has been proposed as a building block to make jet fuel by the Sir Richard Branson Group at Virgin Airlines. Unlike ethanol, butanol does not absorb water and can thus be stored and distributed in the existing petrochemical infrastructure. Due to its higher energy content, the fuel economy (miles per gallon) is better than that of ethanol. Also, butanol-gasoline blends have lower vapor pressure than ethanol-gasoline blends, which is important in reducing evaporative hydrocarbon emissions. It is valuable to fuel refiners/blenders because butanol's low vapor pressure allows the refiner to use more of the light fractions derived from oil refining than is the case when blending gasoline with ethanol. Butanol may be used in precisely the same manner as gasoline, without vehicle modification and without the burden of having to refuel more often.

There is an interest in development of technologies that use renewable resources for energy production. Butanol fermentation is such a process. Traditionally, C. beijerinckii is known for producing n-butanol, ethanol and acetone. Some butyric-acidic-formic acids and yellow oil (a complex mixture of higher alcohols, acids, and esters) are also produced. This organism also produces considerable (and equimolar) quantities of carbon dioxide and hydrogen.

Early in the 20th century, Weizmann discovered a microorganism Clostridium acetobutylicum that was found to possess a remarkable appetite for starch and a still more remarkable ability to convert it into acetone and butanol. See U.S. Pat. Nos. 1,315,585, and 2,386,374.

U.S. Pat. No. 2,420,998 by Beesch and Legg disclosed another organism, Clostridium amylo-saccharobutyl-propylicum, which produced butanol and isopropanol with a small amount of acetone.

U.S. Pat. No. 2,439,791 to Beesch discloses another organism, Clostridium saccharo-acetoperbutylicum, that produced a product similar to that from Clostridium acetobutylicum, but richer in the butanol-to-acetone ratio.

U.S. Pat. No. 5,753,474 (1998), D. E. Ramey, Continuous Two Stage, Dual Path Anaerobic Fermentation of Butanol and Other Organic Solvents using Two Different Strains of Bacteria, describes a process for manufacturing butanol with microorganisms that convert carbohydrates into acids, mainly butyric acid. The acids are subsequently transferred to a different strain of bacteria, which continuously produces butanol and other volatile organic compounds, via a multistage fermentation process. The second microbe has the capability of converting acids into solvents (solventogenesis). The first microbe is now known as Clostridium tyrobutyricum and the second is Clostridium acetobutylicum. By contrast, Clostridium acetobutylicum by itself passes through two separate metabolic cycles. The first cycle is acid-producing (acidogenesis), yielding acetic, butyric, and lactic acids from a carbohydrate source. Then C. acetobutylicum shifts physiology into a solventogenesis phase for the latter part of the cycle, converting the organic acids into acetone, butanol, ethanol, and isopropanol. In summary, two types of microbes were used in two separate processing steps to obtain the final product. See also U.S. Pat. Nos. 1,655,435; 1,818,782; 1,908,361; 2,147,487; and 6,358,717.

Shota Atsumi, Taizo Hanai & James C. Liao, Nature, Vol. 451, pp. 86-90, January 2008, describes the genetic manipulation of E. coli by inserting a first gene that produces aldehydes, and a second gene that converts aldehydes into butanol. The alcohols produced from glucose included isobutanol, 1-butanol, 2-methyl-1-butanol, 3-methyl-1-butanol and 2-phenylethanol.

U.S. Patent Application No. 20090226990 A1 (2008), Andrew C. Hawkins, David A. Glassner, Thomas Buelter, James Wade, Peter Meinhold, Matthew W. Peters, Patrick R. Gruber, William A. Evanko, Aristos A. Aristidou, Methods for the Economical Production of Biofuel from Biomass describe a method of making a biofuel by converting at least two sugars, including a six-carbon sugar or a six-carbon sugar oligomer, and a five-carbon sugar, derived from starch, cellulose, hemicellulose, or pectin.

Published international patent application no. WO/2009/103533 (2009), Gunter Festel, Eckhard Boles, Christian Weber, Dawid Brat, Fermentative Production of Isobutanol using Yeast, describes a process for using genetically-modified yeast to produce isobutanol.

H. A. George, J. L. Johnson, W. E. C. Moore, L. V. Holdeman and J. S. Chen, Applied Environmental Microbiology, Vol. 45-3, pp. 1160-1163, March 1983 describe the screening of thirty-four strains representing 15 species of anaerobic bacteria for acetone, isopropanol, and n-butanol production. Several strains of Clostridium beijerinckii, C. butylicum, and C. aurantibutyricum were reported to produce up to 40-61 mM n-butanol from a 20 g/L glucose feedstock. In these studies the maximum concentration of solvents (butanol & isopropanol) was produced by C. butylicum strain 13,437.

Qurat-ul-Ain Syed, Muhammad Nadeem, Rubina Nelofer, Research Article, Enhanced Butanol Production by Mutant Strains of Clostridium acetobutylicum in Molasses Medium, Vol. 33, Issue 25-30, pp. 25-30, April 2008 describe enhanced n-butanol production by a mutant strain of C. acetobutylicum using blackstrap molasses as substrate. The parent strain C. acetobutylicum PTCC-23 was mutagenized by exposure to UV, N-methyl-N-nitro-N-nitrosoguanidine, and ethyl methane sulphonate and selection for butanol production. The best butanol-producing strain was designated MEMS-7.

Extractive Fermentation—lactic acid and acetone/butanol production, PhD Thesis, Steve Ronald Roffler (1986) describes the use of extractive fermentation to remove inhibitory metabolites from the fermentation broth as they are produced, including the production of acetone and butanol by Clostridium acetobutylicum.

In-situ recovery of butanol during fermentation Part 1: Batch extractive fermentation (1987) pp. 1-12, S. R. Roffler, H. W. Blanch, and C. R. Wilke, Berkeley, reported that end product inhibition could be reduced by the in situ removal of inhibitory fermentation products from an acetone-butanol fermentation. Six solvents or solvent mixtures were tested. The best results were obtained with oleyl alcohol or a mixture of oleyl alcohol and benzyl benzoate.

See also In-situ recovery of butanol during fermentation Part 2: Fed-batch extractive fermentation, Bioprocess Engineering 2 (1987) pp. 181-190, S. R. Roffler, H. W. Blanch and C. R. Wilke, Berkeley; and In Situ Product Removal as a Tool for Bioprocessing, pp. 1007-1012 (1993), BioTechnology 11, Amihay Freeman, John M. Woodley, Malcolm D. Lilly.

See also the review article by D. Jones and D. Woods, Acetone-Butanol Fermentation Revisited, Microbiological Reviews, vol. 50, pp. 484-524 (1986). At p. 515 is a description of solvent extraction of butanol from a fermentation broth using an extractant such as corn oil, paraffin oil, kerosene, or dibutylphthalate.

See also M. Kim and D. F. Day, Butanol production from sugarcane juice, presentation at International Society of Sugar Cane Technology, Mar. 10, 2010 in Veracruz, Mexico.

### SUMMARY

A new strain of Clostridium beijerinckii has been discovered. Fermentation products from the new strain are primarily butanol and isopropanol, with a small amount of ethanol, and little if any acetone. Our novel process has produced up to 29.7% butanol yield from glucose. This butanol-isopropanol mix is easy to recover and is marketable as a mixed alcohol fuel that has the potential of either being used in diesel engines or gasoline engines. Or it can be separated into separate products by way of a low cost distillation.

We have discovered a novel approach to continuously produce and remove butanol from the fermentation broth as it is formed. Higher fermentable sugar levels can be utilized since the butanol concentrations can be maintained below toxic levels. Water usage is also reduced automatically due to the higher permissible sugar levels. For example, if the sugar levels in the feed can be raised from 5 wt % to 10 wt %, it enables a 50% decrease in water usage. With the WEx approach noted below, the resultant water stream can also be recycled economically via a variety of methods.

We have discovered a new fermentation process utilizing carbohydrates as a feed source. This process uses a newly-discovered strain of Clostridium beijerinckii, which is stable in the environment. This fermentation is more economical from a commercial standpoint for the following reasons:

The new, stable strain of Clostridium beijerinckii produces butanol and isopropanol with only small amounts of ethanol, and low or even undetectable amounts of acetone. The mixture of butanol and isopropanol is an extremely versatile mixture for use as flexible fuel. It can be blended into the gasoline pool for use in Otto (spark ignition) engines. It can also be blended into the diesel pool for use in Diesel (compression ignition) engines. Or it may be used as a stand-alone fuel or the components can be separated and used individually.

The fermentation may be conducted in a batch, continuous or semi-continuous manner. The alcohols produced in the broth can be selectively extracted using food grade or non-food grade extractants or mixtures. Using these extractants enables the use of a closed loop multi-component extractant (WEx) system where the extractant loops between a rich mode and a lean mode. Alcohols are removed from the rich WEx stream. The rich WEx stream is converted into a lean stream and recycled. A continuous stream of fermentation broth can be passed through the extractant. The working extractant may, in another mode of operation, be bubbled through the fermenter. The working extractant is chosen so that it is sparingly soluble in water and yet is biocompatible with the fermenting microorganism cultures. The extractant has a high capacity for fermentation products. This property also results in a high disproportionation activity, lower extractant inventory, and a decrease in product recovery costs and complexity. The separation of solvent from an oil emulsion provides a route for low cost, low energy input product recovery.

Theoretically there is a conversion of 61% of carbon atoms to fuel molecules, versus 51% for traditional yeast fermentation to produce ethanol. This differential can provide a substantial economic advantage for a commodity business.

### BRIEF DESCRIPTION OF THE DRAWINGS

FIG. 1 is a graph illustrating solvent production profiles for the test strain.

FIG. 2 is a graph illustrating the production of acetone-butanol, from the individual carbon substrates by C. beijerinckii.

FIG. 3 is a graph illustrating butanol extraction from aqueous phase to oil phase.

FIG. 4 is a graph illustrating butanol extraction from aqueous phase to oil mixtures.

FIG. 5 is a graph illustrating butanol concentration in the presence of various oils.

FIG. 6 is a graph illustrating butanol concentration in the presence of various oil mixtures.

FIG. 7 is a graph illustrating the changes (increases) in butanol yields on fermentation using different oil combinations.

FIG. 8 is a graph illustrating fermentation in the presence of soy oil.

FIG. 9 is a graph illustrating butanol concentration in corn oil phase.

FIG. 10 is a graph illustrating butanol concentration remaining in aqueous phase following extraction with corn oil.

FIG. 11 is a diagram illustrating an example of employing a multi-Component Working Extractant (WEx) Loop.

FIG. 12 is a diagram illustrating another example of employing a WEx Loop.

FIG. 13 illustrates another example to inhibit substrate inhibition of cells.

FIG. 14 is a diagram illustrating another example utilizing immobilized continuous fermenters.

FIG. 15 is a diagram illustrating an example of the extraction of alcohols in the fermentation broth using oleyl alcohol.

FIG. 16 is a diagram illustrating a “dry mill” ethanol production facility.

FIG. 17 is a diagram illustrating the process of dry fractionation.

FIG. 18 is a diagram illustrating an example in which a “dry mill” ethanol production facility is converted into an Optinol production facility.

### DETAILED DESCRIPTION

There are two challenges with butanol fermentation. One is low product concentration and the other is the complexity and cost of separating the various fermentation products from each other. Our novel process solves both problems individually and at the same time. Because butanol is toxic to the producing culture, the maximum concentration of total solvents does not typically exceed 15 g/L in a batch reactor, with a typical weight ratio of 7:3:0.1 Butanol:isopropanol:acetone. Such a low product concentration as well as the mix of fermentation products adversely affects the economics of recovery, due to the need for energy-intensive distillation operations, making the process unable to compete with petroleum-based products. Batch processing generates additional difficulties, including the need for maintenance of strict anaerobic conditions and delicate culture maintenance and propagation protocols. Batch sterilization of large volumes of media is also highly energy intensive.

The Clostridium beijerinckii fermentation production of butanol and isopropanol is an important case where product inhibition affects the overall process. A product, most notably butanol, is toxic to the bacterial cells at the levels produced during the fermentation. Because of such end product toxicity, the final concentration of product on a volume basis is low.

n-Butanol can be produced by Clostridium strains via a pathway that leads from butyryl-CoA to n-butanol. Usually, large quantities of byproducts, such as hydrogen, ethanol, and acetone have been produced in this process, thus limiting the stoichiometric yield of n-butanol to about 0.6 mol of n-butanol per mol of glucose consumed. Historically, two clostridial species, Clostridium acetobutylicum and Clostridium butylicum were the fermentation organisms of choice. In a typical ABE fermentation, butyric, propionic, lactic, and acetic acids are first produced by C. acetobutylicum, the culture pH drops and undergoes a metabolic “butterfly” shift, and butanol, acetone, isopropanol and ethanol are formed. The butanol yield from glucose is low, typically around 15 percent and rarely exceeding 25 percent. By contrast, in preliminary tests our novel process has produced up to 29.7% butanol yield from glucose.

Butanol at a concentration of 1 percent inhibits cell growth and fermentation by about 20%, while 1.6% butanol inhibits growth nearly 100%. The butanol volumetric concentrations produced by conventional ABE fermentations are usually less than 1.3 percent. The butanol yield from glucose is low, typically around 15 percent and rarely exceeding 25 percent by weight. The production of butanol from sugars goes by the biochemical route of producing acetoacetyl-CoA from glucose, and then simultaneously splitting acetoacetyl-CoA to acetone and butyrate with the conversion of butyrate to butanol and, depending on the strain of organism, the conversion of some acetone to propanol. There have been numerous attempts to manipulate the genetics of the organism to separate acetone production from butanol production. These attempts have not been completely successful, as acetone and butanol both share a common intermediate. Attempts to block acetone production to favor butanol production have usually resulted in lower butanol yields.

In one embodiment, there is provided a unique strain of Clostridium capable of metabolizing a carbon source to produce n-butanol and isopropanol with very limited production of acetone and ethanol.

In another embodiment, there is provided a method of producing increased concentrations of n-butanol and isopropanol by growing the organism in the presence of a vegetable oil. The oil acts to sequester some of the solvent from the aqueous phase, decreasing toxicity to the organism and increasing the overall yields.

In yet another embodiment, there is provided a method for recovering n-butanol and other solvent species by collecting emulsion formed from agitating the oil and aqueous growth medium either during or after growth; preferably by continuously separating the emulsion foam; “cracking” the emulsion, for example by using salt, a mechanical breaker, or another emulsification breaking agent known in the art; separating the oil phase from the aqueous phase by decantation; extracting solvent from the oil; and recycling the solvent-depleted oil phase back to the fermentation mixture.

In yet another embodiment, there is provided a method of producing n-butanol and isopropanol, using the bacteria in an immobilized form, comprising (a) culturing to the bacteria on a solid support, placing the support in a column or reactor, then continuously supplying a carbon source, continuously collecting the spent media and then separating the solvents from the spent broth.

In another embodiment, the culture broth is supplied as an emulsion with vegetable oil.

In another embodiment, the culture broth is supplied as an emulsion with corn oil.

In another embodiment, the culture broth is supplied as an emulsion with a mixture of vegetable and mineral oil, such as a mixture of soy oil and mineral oil.

In another embodiment, the emulsion is removed from the fermentation (either at the end of the process or continuously during the process) and broken. Upon breakage two or three layers form, one of which is aqueous and the other(s) are solvent-enriched.

In another embodiment, emulsions with increasing levels of solvent are produced in a sequential multi-stage extraction system using a vegetable oil or vegetable oil mixture. When the solvent concentrations in the emulsion reach a threshold, e.g. 8% v/v, the emulsion is separated and cracked; the phases are allowed to separate; and a solvent-rich fraction is removed by decanting.

Optionally, the bacteria and feedstock are emulsified while charging the fermenter.

Organism

The novel organism is derived from one originally obtained from the Centralbureau voor Schimmelcultures, Utrecht, Netherlands. The accession number of the latter is NCCBNr 84049. The tentative identification was Clostridium sp. Prazmowski 1880 AL. Other identifications were ATCC 27022, NCIB 12605, and strain N1-504. The original isolation was said to be from soil in Japan. It is listed as a source of production of n-butanol and acetone in U.S. Pat. No. 2,945,786. The organism has subsequently been re-identified as Clostridium saccharoperbutylacetonium (Shaheen et al. 2000).

Our analysis of novel organism shows that it is not entirely consistent with any of the above descriptions, and that it is in fact a newly-identified microorganism.

Characterization

C. saccharoperbutylacetonium has been reported to be resistant to rifampin, inhibited by bacteriocin NCP262, and lysogenic to bacteriophage HM7. A DNA sequence is available (Keis et al., 1995 & 2001). This organism was considered to be a phage-resistant strain of ATCC 27021 (Keis et al, 1995).

Biotype differences suggest that the novel organism is actually a separate strain and not a phage resistant mutant of N1-504 (27021). The original strain is rifampin-resistant, will liquefy gelatin (is proteolytic), but does not produce riboflavin. N1-504 will not utilize sorbitol, dulcitol or inositol. Generally strains of C. saccharoperbutylacetonium will utilize arabinose, xylose, glucose, mannose, cellobiose, lactose, maltose, sucrose, starch, glycogen dextrin, pectin and inulin (Keis et al, 2001).

We have found that this new strain does not grow on xylose (Table 1). A full carbohydrate screen was run to test whether the organism is actually N1-504. The organism did not match the reported profile for this organism. Rather it falls closer to (but is not identical with) C. beijerinckii. Table 1 below shows the reported and measured carbohydrate utilization profiles for various strains of Clostridium.

TABLE 1
Carbohydrate utilization by various strains of Clostridium

| | | C. saccharo- | C. saccharo- | |
|---|---|---|---|---|
| | | perbutyl- | perbutyl- | |
| | Novel | acetonium | acetonium | |
| Carbohydrate | organism | N1-4 | N1-504 | C. beijerinckii |
| None (negative | − | − | − | − |
| control) | | | | |
| Glucose | + | + | + | + |
| Mannose | + | + | + | + |
| Arabinose | +/− | + | + | +/− |
| Sorbitol | + | + | − | + |
| Galactose | + | − | − | + |
| Cellobiose | + | + | + | + |
| Xylose | − | + | + | + |

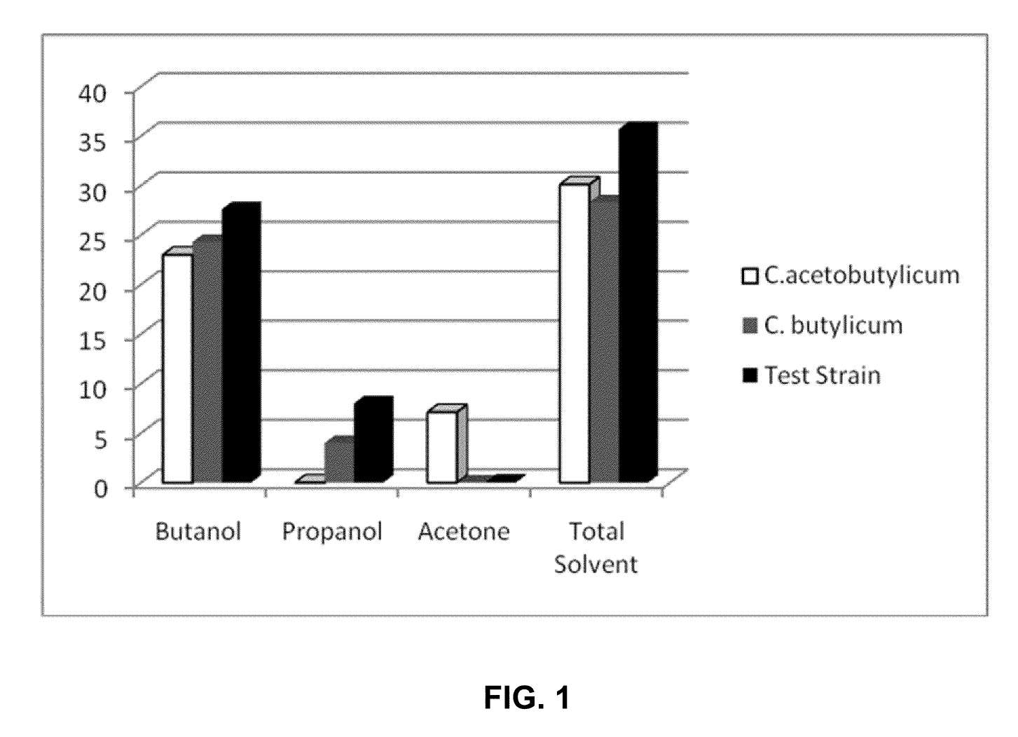

FIG. 1 illustrates solvent production profiles for the test strain. A comparison of reported product profiles for other Clostridia show profiles for the test strain that are closer to C. butylicum than those for C. acetobutylicum. This indicates that the organism this organism behaves most like (but is not identical to) is C. beijerinckii, and is a previously unclassified strain of Clostridium.

A sample of the novel Clostridium strain, designated Clostridium beijerinckii sp. optinoii, was deposited with the American Type Culture Collection (ATCC), 10801 University Boulevard, Manassas, Va. 20110-2209, United States on Sep. 8, 2010 2010, and was assigned ATCC Accession No. PTA-11285. This deposit was made under the Budapest Treaty.

The handling of the culture is important. In a specific process, a first step is to remove some of the spores and add them to a sterilized glucose medium. The latter is then heat-shocked by placing it in boiling water for 90 seconds, removing, and cooling immediately to 30° C. The heat shock stimulates the spores, causing germination and, at the same time, eliminates the weaker spores. After the heat shock the culture is incubated at 30° C. for 20 to 24 hours. At the end of 20 to 24 hours the culture is transferred aseptically to 600 ml. of sterilized molasses mash of the following composition: 4% sugar (supplied in the form of invert molasses), 5% ammonium sulfate, 6% calcium carbonate, and 0.2% phosphorus pentoxide (supplied in the form of superphosphate. (The amounts of the various chemicals are all based on the weight of the sugar used.) After transfer the culture is plated out to detect any aerobic contaminants.

Yields

Media Composition

Basal medium comprised a carbon source (variable) supplemented with 1 g/L yeast extract; KH₂PO₄, 5 g/L; K₂HPO₄, 5 g/L; ammonium acetate, 22 g/L; MgSO₄.H₂O, 2 g/L; MnSO₄, 0.1 g/L; FeSO₄.7H₂O, 0.1 g/L; NaCl, 0.1 g/L; p-aminobenzoic acid, 0.001 g/L; thiamin, 0.01 g/L; and biotin, 0.0001 g/L. The pH was adjusted to 6.4, and growth was conducted at 34° C.

Product Yields: C Source

The product yields for N-504 have been reported to reach 9.8 g/L of solvent in a synthetic medium and 15.6 g/L in a molasses medium containing 6% fermentable sugars (Shaheen et al, 2000). They also reported that many strains cannot utilize molasses when the concentration of fermentable sugars exceeds 6-6.5%.

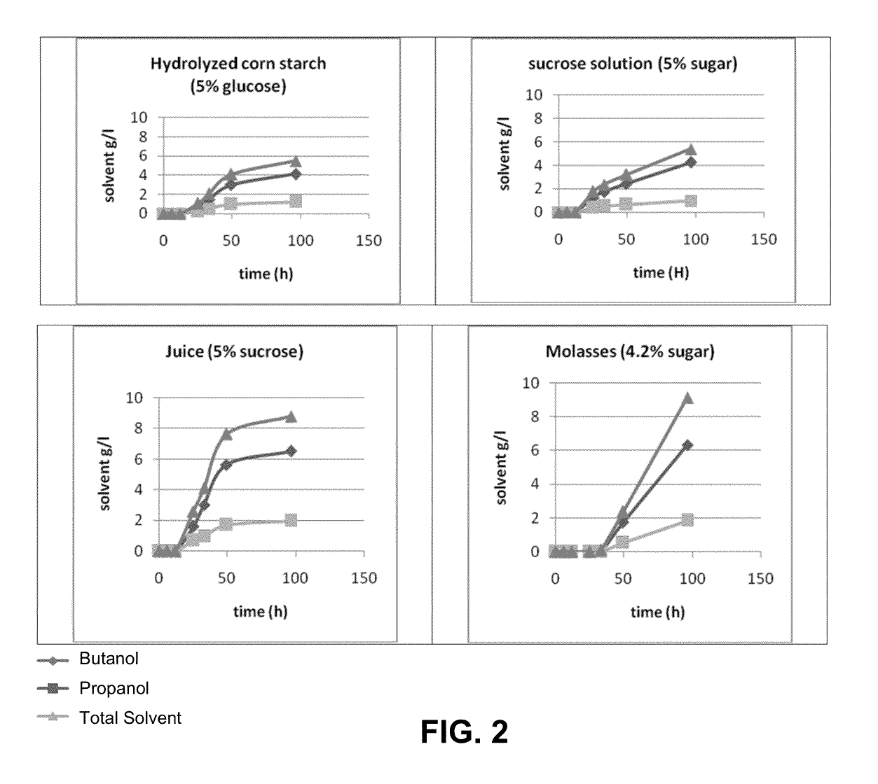

FIG. 2 illustrates the production of acetone-butanol, from the individual carbon substrates by C. beijerinckii. FIG. 2 and Tables 2-5 show the yields and composition obtained for “Optinol”, the solvent mix obtained, from various carbon sources using the novel Clostridium strain. The glucose was that obtained from corn starch hydrolysis. (These yields were not optimized; it is likely that by varying composition and conditions appropriately, higher optimized yields may be possible for each of the carbon sources.)

TABLE 2
Solvent yields as a function of feedstock (g/L)

| C-source | % | acetone | Ethanol | isopropanol | n-butanol | Total |
|---|---|---|---|---|---|---|
| Glucose | 5.1 | — | 0.08 | 0.51 | 2.24 | 2.83 |
| Corn starch | 5.0 | 0.02 | 0.13 | 1.21 | 4.12 | 5.48 |
| Sucrose | 3.8 | 0.01 | 0.11 | 0.52 | 2.6 | 3.22 |
| Sucrose | 5.0 | 0.03 | 0.14 | 0.96 | 4.3 | 5.39 |
| Molasses | 1.9 | — | 0.086 | 0.36 | 2.60 | 3.05 |
| (cane) | | | | | | |
| Molasses | 4.2 | 0.08 | 0.18 | 1.84 | 6.33 | 8.43 |
| (cane) | | | | | | |
| Cane Juice | 5.0 | 0.02 | 0.13 | 1.21 | 4.12 | 5.48 |

Growth proceeded to exhaustion (96 hrs). The growth curves showed that the solvent production followed growth, peaking when growth stopped. The resulting mixture of solvents will sometimes be referred to as “Optinol.”

TABLE 3
Productivity during log growth phase

| | Solvent Produced | Maximum Productivity Achieved |
|---|---|---|
| | (Volumetric | Over The Log Growth Phase |
| C-source | %/hr) | (g solvent/g sugar) |
| Glucose (starch) | 0.53 | 0.47 |
| Sucrose | 0.53 | 0.56 |
| Cane juice | 0.85 | 0.67 |
| Cane Molasses | 0.85 | 0.63 |

TABLE 4
Specific yields as a function of C-source

| | | Sugar | Solvent | Yield (grams of |
|---|---|---|---|---|
| | | consumed | (“Optinol”) | solvent per gram |
| | % | (g/L) | produced (g/L) | of feedstock) |
| Glucose | 5.1 | | | |
| Corn starch | 5.0 | 14.97 | 5.48 | .37 |
| Sucrose | 3.8 | 13.87 | 3.22 | .23 |
| Sucrose | 5.0 | 16.03 | 5.39 | .34 |
| Molasses (cane) | 1.9 | | 3.05 | |
| Molasses (cane) | 4.2 | 21.98 | 8.43 | .38 |
| Cane juice | 5.0 | 20.06 | 8.8 | .44 |

TABLE 5
Optinol Composition (%) as a function of carbon source

| C-source | % | acetone | ethanol | i-propanol | n-butanol |
|---|---|---|---|---|---|
| Glucose | 5.1 | — | 3.0 | 18 | 79 |
| Corn starch | 5.0 | 0.4 | 2.3 | 22.1 | 75.2 |
| Sucrose | 3.8 | 0.4 | 3.5 | 16.0 | 80.0 |
| Sucrose | 5.0 | 0.5 | 2.6 | 17.8 | 79.1 |
| Molasses(cane) | 1.9 | — | 2.8 | 11.8 | 85.2 |
| Molasses(cane) | 4.2 | 0.9 | 2.1 | 21.8 | 75.1 |
| Cane juice | 5.0 | 0.7 | 2.9 | 22.3 | 74.0 |

Regardless of the carbon source, the Optinol product composition did not vary widely. In these prototype runs, the composition of butanol was ˜75-85%, of isopropanol was ˜12-22%, of ethanol was ˜2-3%, and of acetone was ˜0.4-0.9%.

Other potential carbon sources are those known in the art including, for example, corn syrup, sweet sorghum juice, sugar beet molasses, cheese whey, and corn steep liquor.

Production

In Situ Extraction

A major problem blocking increased production of solvents in traditional ABE fermentation has been the toxicity of the solvent to the organism and the complexity and cost of separating the fermentation products from each other. Butanol dissolves cell membranes. Commonly levels of butanol do not exceed ˜16 g/L before the fermentation broth becomes toxic to the organism. In one embodiment of the novel process, solvent is removed as it is produced (in situ), lowering the concentration of butanol in the aqueous phase. The method employs intimately mixing the fermentation culture with a non-toxic, hydrophobic component (e.g. a vegetable oil or vegetable oil mixture, for example corn oil or a mixture of oils containing corn oil.)

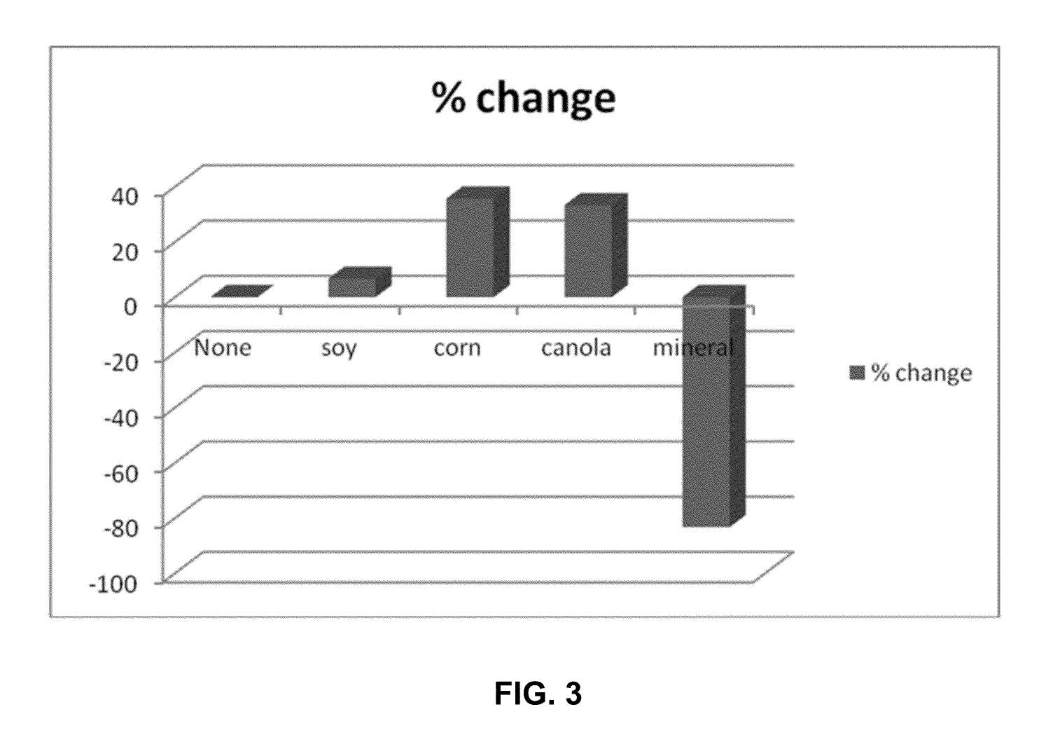

FIG. 3 illustrates butanol extraction from aqueous phase to oil phase. The hydrophobic component extracts solvent during production, sequestering it from the organisms. This approach allowed overall solvent concentrations of 20 g/l or higher. A screen of different oils showed that both corn and canola oils improved butanol products and increased total solvent yield ˜35%. NOTE: The data shown in FIG. 3 through FIG. 7 depict only the butanol component. Isopropanol and ethanol were not separately measured in the experiments that generated the data.

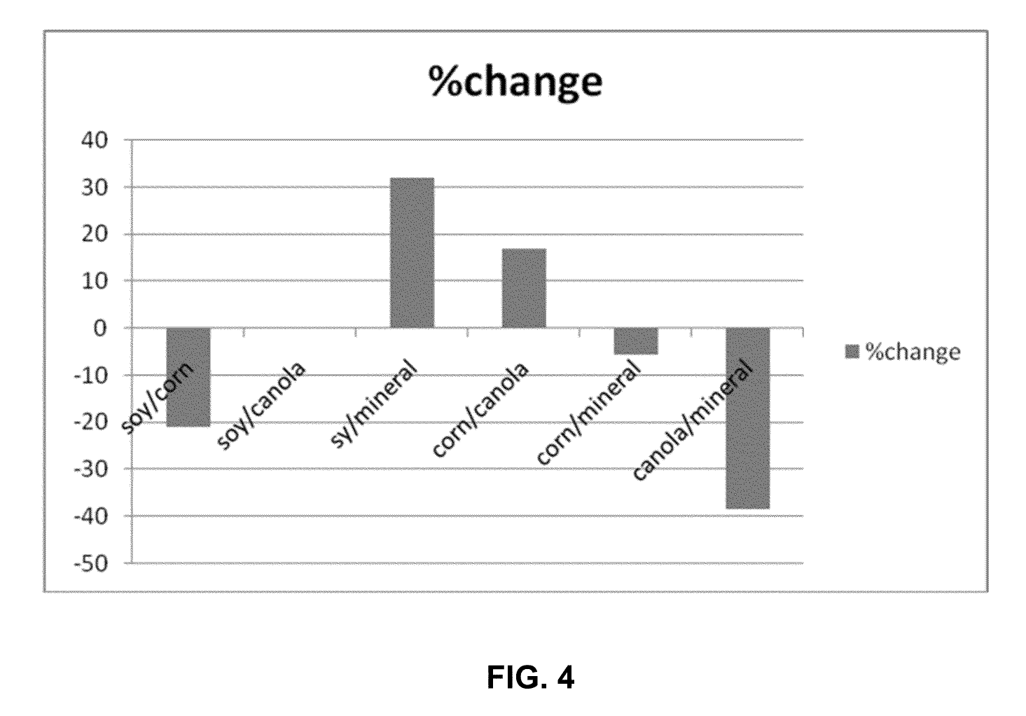

FIG. 4 illustrates butanol extraction from aqueous phase to oil mixtures. Although mineral oil caused a decrease in yield, and soy oil improved yield far less than either corn oil or canola oil, a mixture of soy oil and mineral oil gave an even higher yield than either corn oil or canola oil alone. FIG. 4 shows the changes in butanol concentration obtained with 1:1 oil mixtures (20% V/V, on fermentation broth) as compared to the best results obtained with corn oil (20% v/v; FIG. 3). (Corn oil alone, not depicted in FIG. 4, would represent ˜35.7%.) The use of soy/mineral oil mixtures theoretically should give a butanol concentration in the fermentation broth of 26 g/l, a factor of 1.6 times higher than achieved in fermentation without oil.

Screening of Vegetable Oils in Fermentation

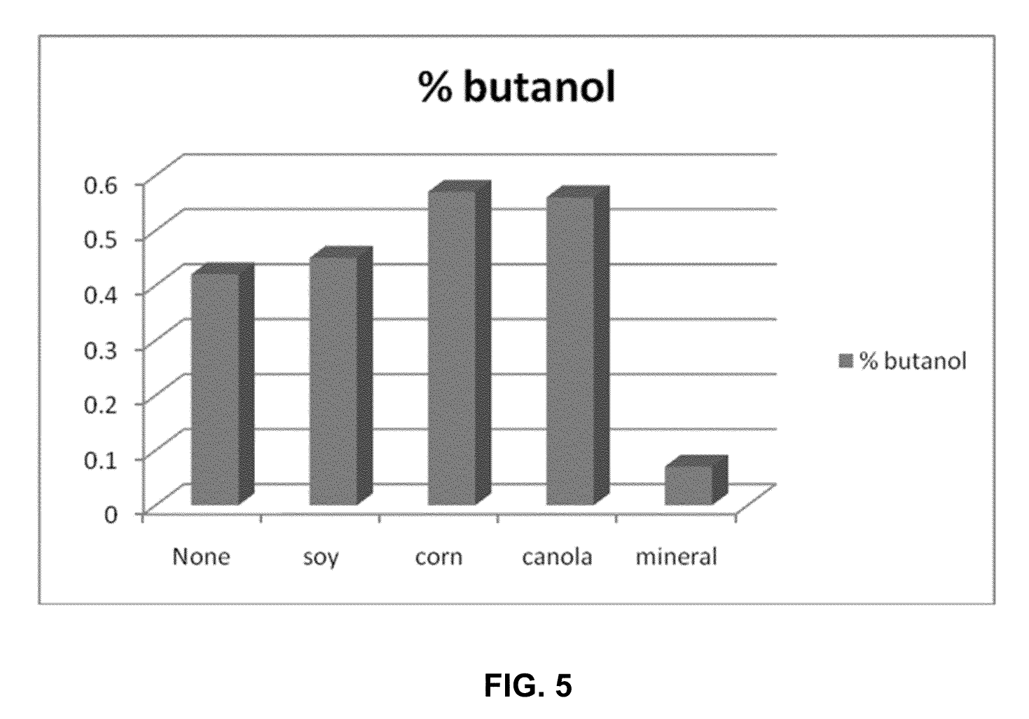

FIG. 5 illustrates butanol concentration in the presence of various oils. A series of static fermentations topped with a 20% v/v oil layer were conducted. A screen of single oils at 20% v/v showed that corn and canola oils gave the best results in terms of increasing yields. The use of mixed (50:50) oils (20% v/v) gave a different pattern.

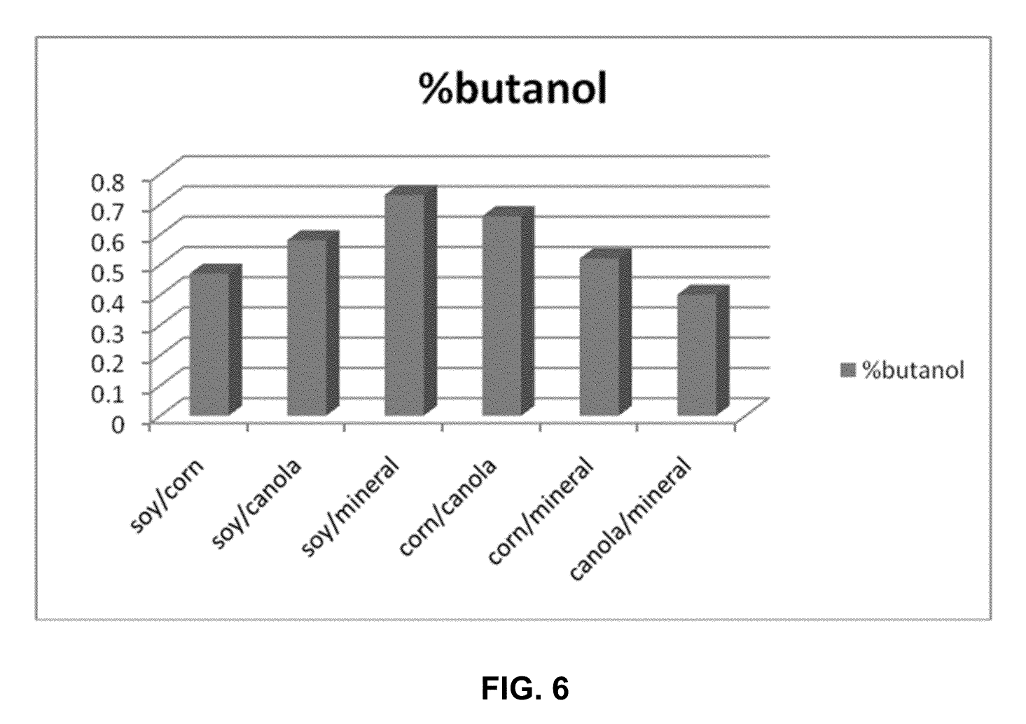

FIG. 6 illustrates butanol concentration in the presence of various oil mixtures. There was an increase in butanol yields with the soy and canola oil mixture over what was seen with soy oil only. However, the levels produced with this mixture were no better than what could be achieved with canola oil alone. Surprisingly, the soy and mineral oil mixture produced the greatest increase in butanol production as compared to fermentation without oil.

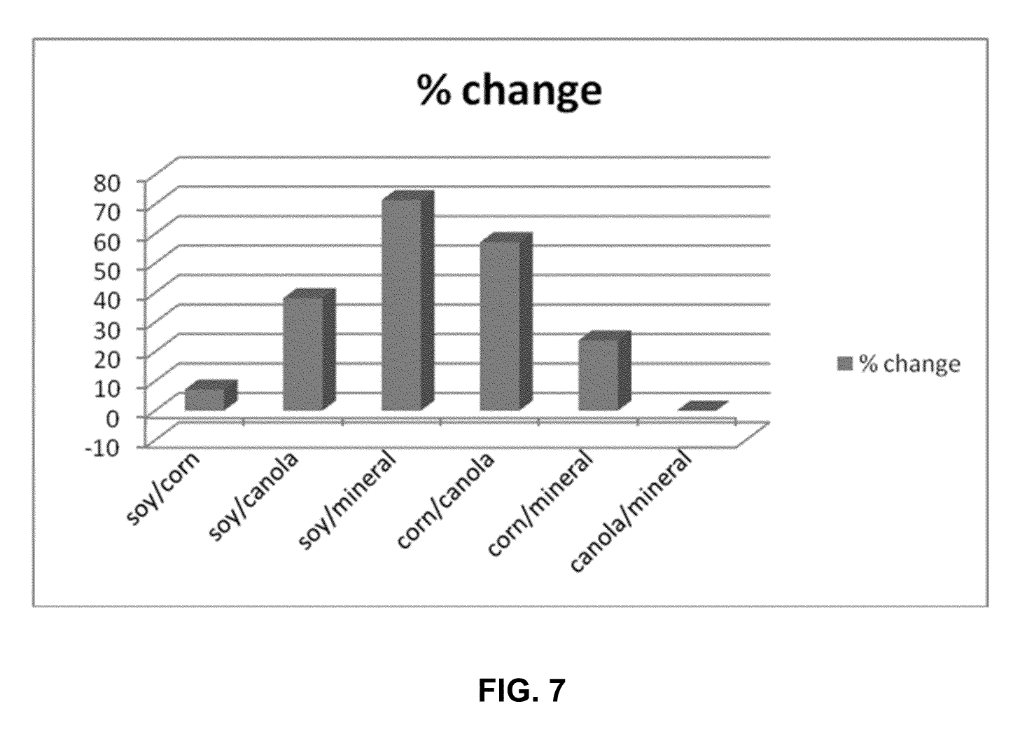

FIG. 7 illustrates the changes (increases) in butanol yields on fermentation using the different oil combinations. Because mineral oil alone inhibits butanol production, the enhanced yield with a mixture including mineral oil was quite surprising. Canola alone substantially enhanced yield; but mixing mineral oil with canola oil effectively neutralized the yield enhancement from canola. Thus it was especially surprising that adding mineral oil to a different oil (soy oil) substantially enhanced the yield. These results have been replicated, and appear to be robust.

It is possible that the degree of unsaturation of the various oils may account for some of these observations, as one would expect better solubility with a more unsaturated oil, but one would still not expect the results seen with the soy/mineral oil mix. Soy has the same degree of unsaturation as canola, while mineral oil is not saturated.

Use of Soy Oil in Fermentation

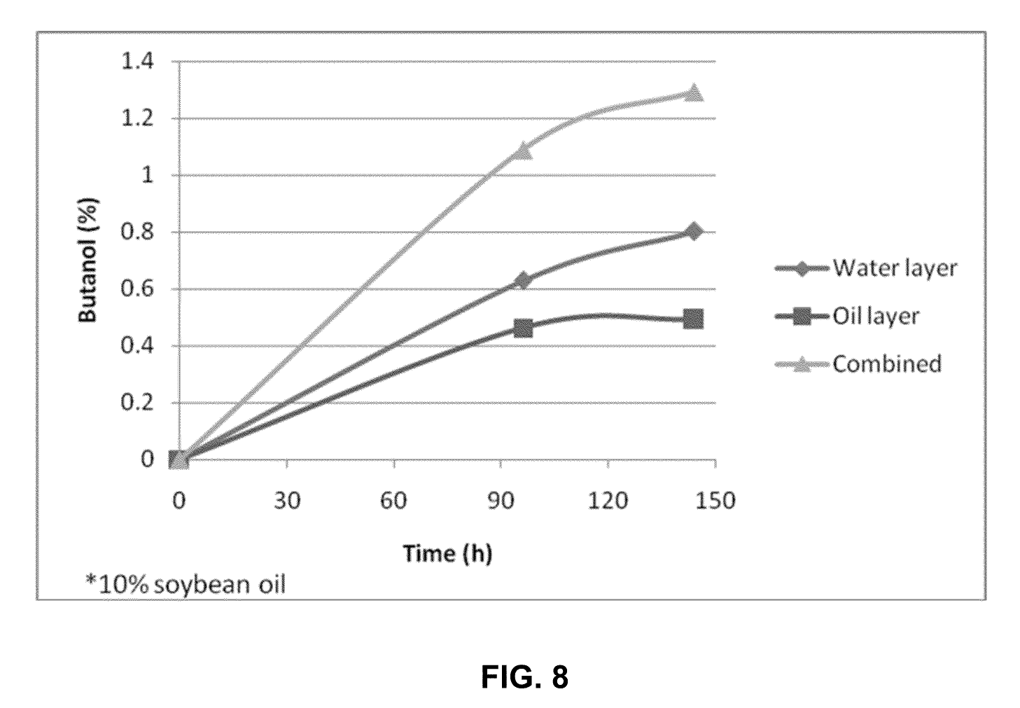

FIG. 8 illustrates fermentation in the presence of soy oil. Soy oil (10% v/v) in fermentation showed the oil layer saturated with butanol after 90 hours of fermentation.

Extractions-Product Recovery

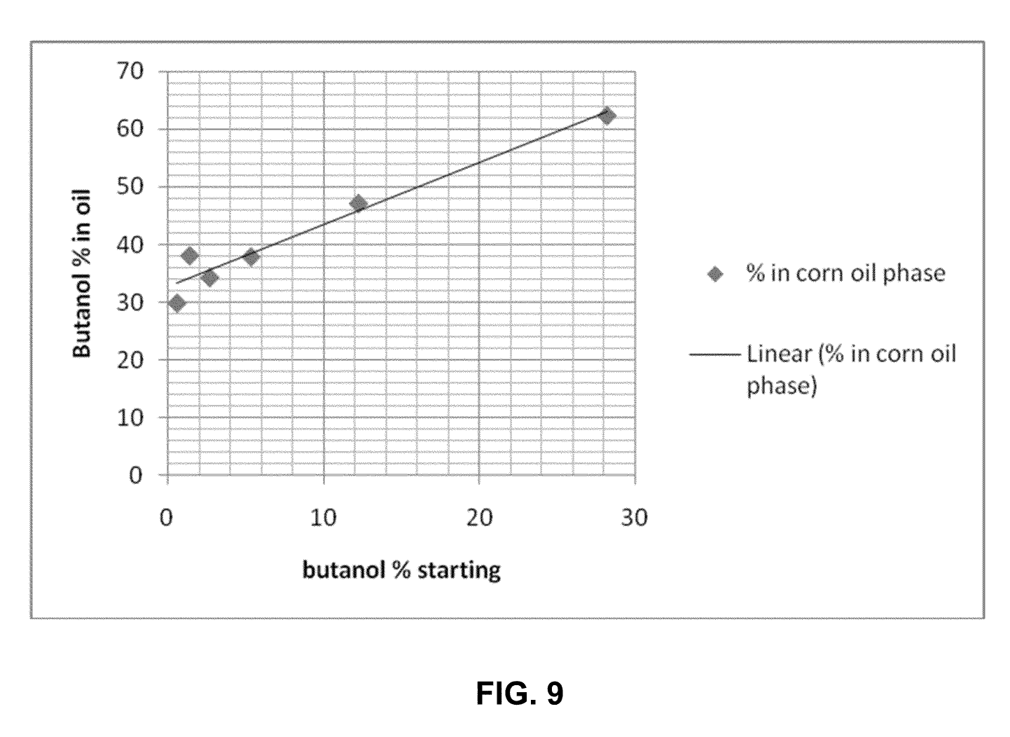

FIG. 9 illustration butanol concentration in corn oil phase. Extractions with a 20% v/v corn oil, against water/butanol mixtures containing varying amounts of butanol, showed a nearly linear increase in the amount of butanol in the oil with increasing butanol in the aqueous phase (FIG. 7). These were shaken extractions (1 min).

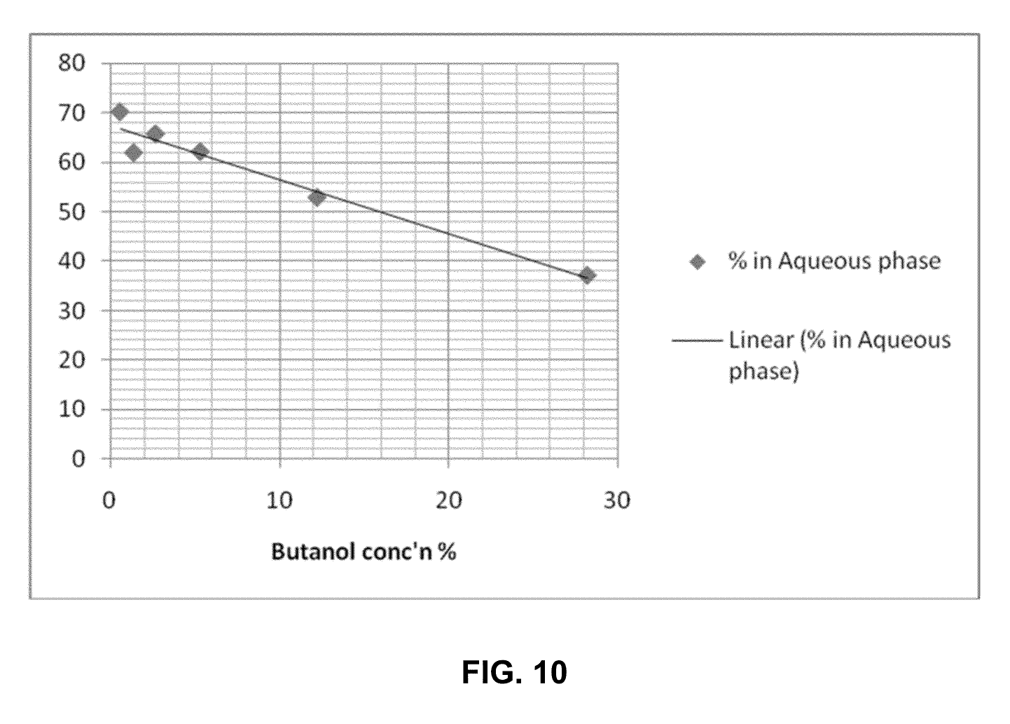

FIG. 10 illustrates butanol concentration remaining in aqueous phase following extraction with corn oil. Conversely, the amount of butanol remaining in the aqueous phase following extraction with 20% v/v corn oil, dropped linearly with the starting concentration of butanol.

For example, at a 16 g/L butanol starting concentration, we can remove 34% of the butanol from solution. This indicates that it may be possible, for example, for an organism that normally produces 16 g/L butanol, instead to produce 21 g/L in the presence of oil in a batch production system.

There are at least two approaches for using oil to enhance the yield of the novel process. The use of oil as part of the fermentation, with continuous removal of oil from the system, will give higher yields of solvent. An aqueous/oil emulsion forms during the fermentation, and typically produces substantial amounts of foam. The amount of foam varies with the degree of stirring. The foam can be removed from the fermentation, and then the foam may be “broken” by any of several methods known in the art. Among the methods for “breaking” a foam emulsion are chemical (e.g., the addition of a salt such as sodium chloride or ammonium sulfate), or an antifoaming agent (e.g., polypropylene glycol, various silicones, or polyglycols), or mechanical (e.g., a foam control device or foam breaking device).

We have also conducted tests using an artificial 80:20 (v/v) mixture of water and oil, with varying amounts of butanol. With starting concentrations of 0.5%, 1% and 2% butanol (v/v), there were two visible layers following “breaking”: a water layer, and a water-oil layer. With butanol concentrations of 4% or higher there was a larger volume emulsion. At 8% butanol, there was still an emulsion, but also a small layer of butanol formed on top of the oil. With 16% butanol, the emulsion layer disappeared and there was a distinct butanol layer on top of the oil.

An example of a method of producing butanol is to sequentially or concurrently: conduct the fermentation, mix the fermentation mixture with oil, skim the foam, break the foam to separate solvent, and recycle the oil for further use. This method may be used in batch mode, or preferably, in continuous mode. Several prior references, in describing other butanol fermentation processes, have observed that foaming is undesirable; or that antifoaming agents need to be applied; or that there are detrimental effects from antifoaming agents—to the effect that the use of antifoaming agents was undesirable but reflected a necessary compromise. These references suggest that foaming is undesirable, or that it is preferred to inhibit foaming. The process may or may not avoid foaming, but in a specific implementation in which the process does not avoid foaming, the process can affirmatively make beneficial use of the foam generated by fermentation. Thus the process proceeds in a direction opposite from that suggested by the prior references. We found that the alcohols are more highly concentrated in the foam, and that it is actually more efficient to separate the alcohols from the foam than from the liquid phase.

A second approach to extraction of solvent from the aqueous phase is to use oil as an extraction fluid in a multi-stage extractor. When the butanol concentration reaches a concentration above ˜8%, the foam is broken, and the solvent is separated by decanting. This is approach should reduce energy consumption. Conversely, the oil used during fermentation can be recycled as an extractant in a liquid-liquid separation to reduce the energy cost of producing Optinol from the fermentation.

Product Variability and Fermentation Repeatability

A series of stirred fermentations was conducted using a corn oil layer (20% v/v). Solvent concentrations were determined at the end of a 144-hour fermentation. Fermentations (3 L) were conducted in duplicate. Butanol levels were measured in the aqueous and oil layers.

TABLE 6
Solvent Profile-duplicate stirred fermentations,
numbers are in %, +/−SD (n = 2)

| | | | Total Solvent in |
|---|---|---|---|
| Solvent | Aqueous layer | Oil layer | both layers (%) |
| Acetone | 0.013 (.001) | 0 | 0.013 |
| Ethanol | 0.025 (.003) | 0 | 0.025 |
| Isopropanol | 0.295 (0.35) | .0275 (.0007) | 0.3225 |
| n-Butanol | 0.74 (.08) | 0.491 (0) | 1.231 |

Effect of Stirring on Butanol Yields

Matched three liter fermentations of glucose were run, with and without stirring. Butanol levels were determined in the aqueous and oil layers.

TABLE 7
Role of stirring with oil extraction on butanol yields

| Layer | Stirred (g/L) | Unstirred (g/L) |
|---|---|---|
| Aqueous (80% of volume) | 6.85 | 4.58 |
| Oil (20% of volume) | 3.94 | 2.78 |
| Total (per liter volume) | 6.27 | 4.22 |

Stirring produced a 47% increase in the total amount of butanol. These observations support our conclusion that that continuous extraction of the solvent enhances total production yields.

Scalability-Comparison Fermentations

Fermentation (96 hr) was performed in a 60 L container (one run only) containing 20% corn oil and 5% glucose as the carbon source. There was no flushing of the fermentation with nitrogen gas (i.e., the fermentation was not strictly anaerobic). These conditions led to an environment more likely to favor mixed acid strain contaminants, which can produce ethanol. We believe that efficiency upon scale-up will be improved by promoting strictly anaerobic fermentation conditions at the larger scale.

TABLE 8
Solvent composition in corn oil layer (g/L)

| Solvent | 3 L Fermentation | 60 L Fermentation |
|---|---|---|
| Acetone | 0 | 0.05 |
| Ethanol | 0 | 0.19 |
| Iso propanol | 0.275 | 0.26 |
| n-Butanol | 4.91 | 3.42 |

Vacuum Extraction

Following the 60 L fermentation, solvents in the oil layer were extracted using a vacuum evaporator at 117-120° C. This particular procedure preferentially concentrated the ethanol and isopropanol over the butanol.

TABLE 9
Solvent composition of initial oil layer and of distillate

| | Solvent | | | |
|---|---|---|---|---|
| | concentration (g/L) | | | Boiling |
| | Initial Oil | | Concentration Factor | point |
| | layer | Distillate | (distillate:initial ratio) | (° C.) |
| Acetone | 0.05 | 0.59 | 12 | 56.5 |
| Ethanol | 0.19 | 13.41 | 71 | 78.4 |
| Isopropanol | 0.26 | 15.59 | 60 | 82 |
| n-Butanol | 3.42 | 62.56 | 18 | 117 |
| Total | 3.92 | 92.14 | | |
| Corn oil | | | | 216 |

Accumulation of product(s) in fermentation broths can inhibit productivity. This phenomenon, if not addressed properly, can be a major drawback in the efficiency and economics of fermentation technologies.

In the fermentation, the n-butanol and 2-propanol accumulate and eventually inhibit the overall process. The product, notably butanol, is toxic to the bacterial cells at the levels produced during the fermentation, before complete conversion of the typical feedstock. The inhibition levels range in concentration levels from 10-15 g/L (1.5-2.0 wt %). One way to avoid such end-product toxicity is to keep the initial concentration of fermentable sugars relatively low, about 5.0-6.5 wt %.

Another approach is to continuously remove butanol from the fermentation broth as it is formed. Higher fermentable sugar levels can be utilized if the butanol concentrations are kept below levels that could be toxic to the bacteria. Water utilization levels can also be reduced automatically due to the higher permissible sugar levels. For example, if the sugar levels in the feed can be raised from 5 wt % to 10 wt %, water usage can decline by 50%.

Liquid-Liquid Extraction

Liquid-liquid extraction is a process for separating components in solution by their distribution between two immiscible liquid phases. Typically the feed to a liquid-liquid extraction process is a solution that contains the components to be separated. Minor components in the feed solution are referred to as solutes. The extraction solvent is the immiscible liquid added to the process for extracting the solutes from the feed. An equilibrium stage is a combination of operations that accomplishes the effect of intimately mixing two immiscible liquids until equilibrium concentrations are reached and then physically separating the two phase into distinct layers. Countercurrent extraction is a procedure where the extraction solvent enters a stage furthest from where the feed enters. The two phases then pass each other in a countercurrent fashion. The objective is to transfer one or more components from the feed into the extractant, usually in a number of stages (typically about a dozen or so). At each stage, the two phases are intimately mixed, with droplets of one phase suspended in the other; and then the phases are separated each time before they are transferred to the next stage in countercurrent fashion.

Liquid-Liquid Extraction with Corn Oil

Two runs were conducted, each with a different flow rate.

TABLE 10
Flow Rates (ml/min) for Karr Extractor

| Run # | Optinol In | Oil In | Extract Out | Raffinate Out |
|---|---|---|---|---|
| 1 | 26 | 7 | 5.7 | 26.8 |
| 2 | 39 | 7 | 5.3 | 40 |

Partition Data

TABLE 11
Product Partitions (run #1)

| | | Extract out | Raffinate out | |
|---|---|---|---|---|
| Product | In (mg) | (mg) | (mg) | Recovery (%) |
| Optinol | 314.1 | 33.8 | 318.4 | 112 |
| butanol | 252.7 | 29.5 | 250.6 | 111 |
| Isopropanol | 61.4 | 4.3 | 67.8 | 117 |

TABLE 12
Product Partitions (run #2)

| | | Extract out | Raffinate out | |
|---|---|---|---|---|
| Product | In (mg) | (mg) | (mg) | Recovery (%) |
| Optinol | 471.1 | 25.0 | 446.1 | 100 |
| butanol | 379.1 | 21.7 | 354.9 | 99.3 |
| Isopropanol | 92.0 | 3.33 | 91.2 | 102.7 |

TABLE 13
Extraction Percent by Flow Rate

| | Run 1 | Run 2 |
|---|---|---|
| Flow rate feed ml/min | 26 | 39 |
| Optinol % extraction | 33.3 | 29.7 |
| Butanol % extraction | 35.6 | 31.5 |
| Isopropanol % extraction | 23.0 | 21.5 |

Comments: Butanol extraction is favored. It is running around 30% in this set up. Isopropanol extraction is about 21%

Liquid Liquid Extraction with Oleyl Alcohol

The extraction was conducted with room temperature separation.

TABLE 14
Feed Table

| | | |
|---|---|---|
| Raffinate | Butanol | 126.36 mg |
| | i-Propanol | 30.68 mg |
| | water | 13,000 mg |
| Oleyl Alcohol (20,000 mg) | Butanol | 0 mg |
| | i-Propanol | 0 mg |
| | Water | 0 mg |

TABLE 15
Effluent Table

| | | |
|---|---|---|
| Raffinate | Butanol | 8.86 mg |
| | i-Propanol | 19.95 mg |
| | water | 12,400 mg |
| Oleyl Alcohol (20,000 mg) | Butanol | 117.5 mg |
| | i-Propanol | 10.73 mg |
| | Water | 600 mg |

The above table shows separation compositions for Optinol at a 1.5 Solvent: Feed ratio. The values are given as mg of material (independent of flow rate). The water value is an estimate, based on water not recovered, of water in the Oleyl alcohol mixture.

It is obvious that with the appropriate choices liquid-liquid extraction can be used to increase the concentration of product in the raffinate such that the ability to use distillation as a final recovery is enhanced.

Attributes of specific implementations of Multi Component Working Extractant (WEx) are discussed below:

Reduced end-of-production inhibition: a consequence of extracting toxic fermentation products (butanol, butyric acid, etc.) from the broth into the extractant.

The extractant should be bio-compatible with the fermenting microorganism.

The extractant should be sparingly soluble in water to minimize extractant losses.

Traces of residual extractant in the broth should not affect the values or efficacy of stillage byproducts.

The extractant should have a high affinity for and capacity for the fermentation solvent products. This property results in:

High disproportionation activity

Lowered extractant inventory

Decreased product recovery costs and complexity

Extractant should possess a low vapor pressure to minimize losses in the alcohol stripping column

The release activity of the extractant-alcohol mixture should allow the use of low pressure steam in the reboiler of a stripping column.

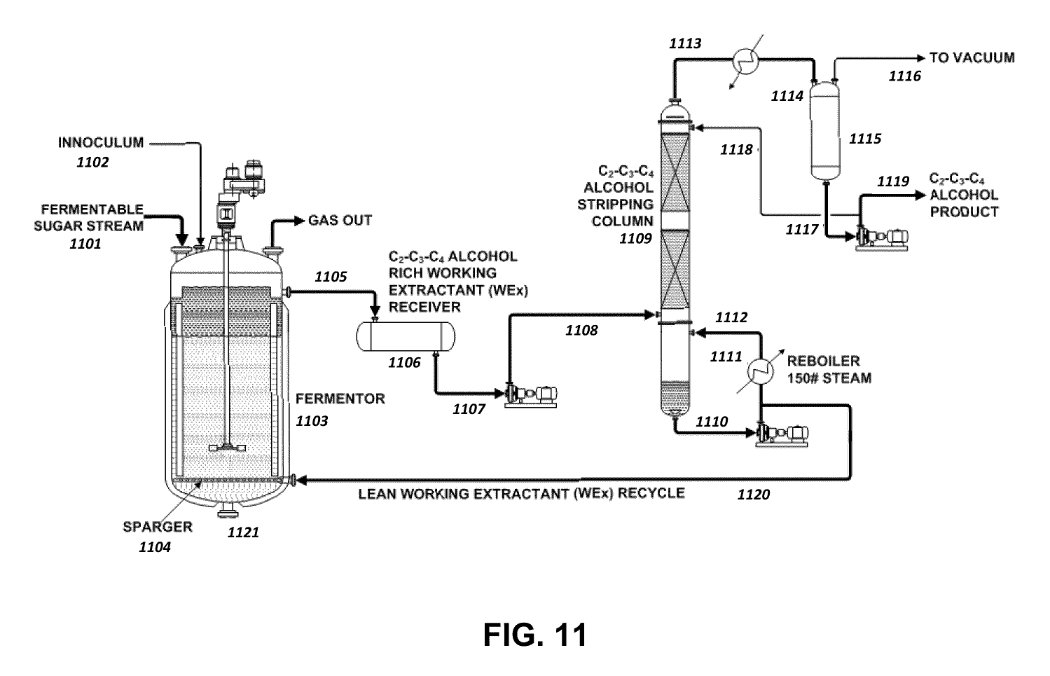

FIG. 11 illustrates an example of employing a multi-Component Working Extractant (WEx) Loop. A fermentable sugar stream 1101 is introduced into the fermenter as a fed-batch operation. This technique is used to inhibit substrate inhibition of the cells by high concentrations of sugar in the fermenter. The inoculum 1102 is introduced into the fermenter during the initial fill-up stage. Fermentation is conducted in the fermenter 1103 and yields a broth that includes the C₂—C₃—C₄ alcohol products, comprising primarily the C₄, or butanol fraction.

The working extractant (WEx) loop uses anedible, water-immiscible oil as the extractant. In the working loop, the oil is sparged into the bottom of the fermenter via sparging ring 1104. Fine droplets of oil float upwards through the fermentation broth and absorb alcohols from the fermentation broth.

The upper section of the fermenter is equipped with an overflow well that collects the C₂—C₃—C₄ alcohol-rich, working extractant solution. The rich working extractant 1105 overflows into collecting receiver 1106. It is also possible to use a number of fermenters working simultaneously, to flow into a common receiver. The rich working extractant stream 1107 flows out of the receiver into a pump which takes the rich working extractant solution 1108 and directs it into alcohol stripping column 1109.

The alcohol stripping column is designed to take the bottoms 1110 in the column and pump them through a low pressure steam reboiler 1111 under sufficient vacuum to strip out the alcohols. The liquid exiting the reboiler 1112 is recirculated back into the bottom of the column. The stripped vapors from the top of the column 1113, comprising primarily the C₂—C₃—C₄ alcohols, flow through a condenser. The condensed liquid 1114 is collected in an overhead receiver 1115 and appropriate recycles are maintained to ensure product purity. Vacuum 1116 is maintained to ensure appropriate vapor velocities within the stripping column. Liquids from the overhead receiver 1117 flow into a pump where a part of the pumped stream 1118 is refluxed back into the column, and the balance is pumped out as C₂—C₃—C₄ alcohol product 1119. The overhead alcohol product (C₂—C₃—C₄ alcohol) is pumped to storage tanks.

The bottom of the alcohol stripping column has a reboiler tied to a recycle stream. An appropriate bottoms recycle is maintained to ensure a low residual level of alcohol in the working extractant. This stream, referred to as the lean working extractant 1120, is directed to the sparger at the bottom of the fermenter, closing the loop.

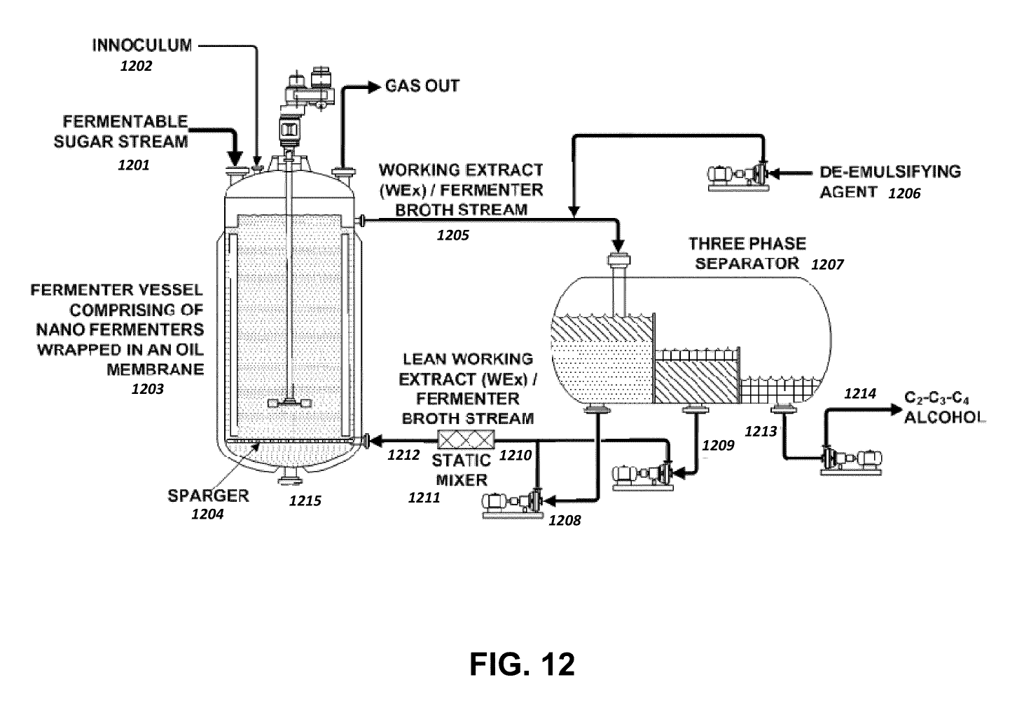

FIG. 12 illustrates another example of employing a WEx Loop. Fermentable sugar stream 1201 and inoculum 1202 are introduced concurrently into a fermenter vessel. The fermenter vessel 1203 comprises nanofermenters, in which the organisms and fermentable sugars are encapsulated within an oil membrane. The fermentation broth within the nano fermenters comprises includes the C₂—C₃—C₄ alcohol product, comprising primarily the C₄, or butanol fraction. A sparger 1204 is incorporated in the bottom of the fermenter to introduce the lean working extract and fermenter broth. This results in a continuous upflow of product within the fermenter vessel.

A composite stream comprising the working extractor (WEx) and the nanofermenters exits the fermenter 1205. A de-emulsifying agent 1206 is pumped into this stream, and the combined product then flows into three-phase separator 1207. The three-phase separator vessel comprises three distinct compartments separated by walls that decrease in height, to control the residence time of liquids within the compartments. The working extract, the nanofermenters and the de-emulsifying agents flow into the first compartment. Separation occurs, and the fermenter broth settles to the bottom of the compartment while the extractant and the C₂—C₃—C₄ alcohol product overflow the side wall of this compartment. The recovered fermenter broth 1208 is pumped back into the fermenter vessel. The oil and alcohol overflow into the second compartment, where the de-emulsifying agent separates the oil and the alcohol into two separate phases. The broth separates from oil nanospheres in the first compartment. The de-emulsifying agent then ruptures the oil nanospheres in the next compartment, to separate the oil phase and the alcohol phase. The oil phase 1209 settles to the bottom of the compartment, and this stream is pumped back and merged into the lean working extract stream 1210. The broth and lean working extract stream flow into static mixer 1211, and the output of this mixed composite stream 1212 is introduced back into the fermenter through the sparger.

The overflow from the second compartment goes into the third compartment of the three-phase separator. This overflow comprises the C₂—C₃—C₄ alcohols, and is withdrawn from the compartment under level control. This stream 1213 is pumped out as C₂—C₃—C₄ alcohol product 1214. The alcohol-depleted fermentation broth 1215 is sent to another fermentation operation, where the residual alcohol is converted into an amino acid.

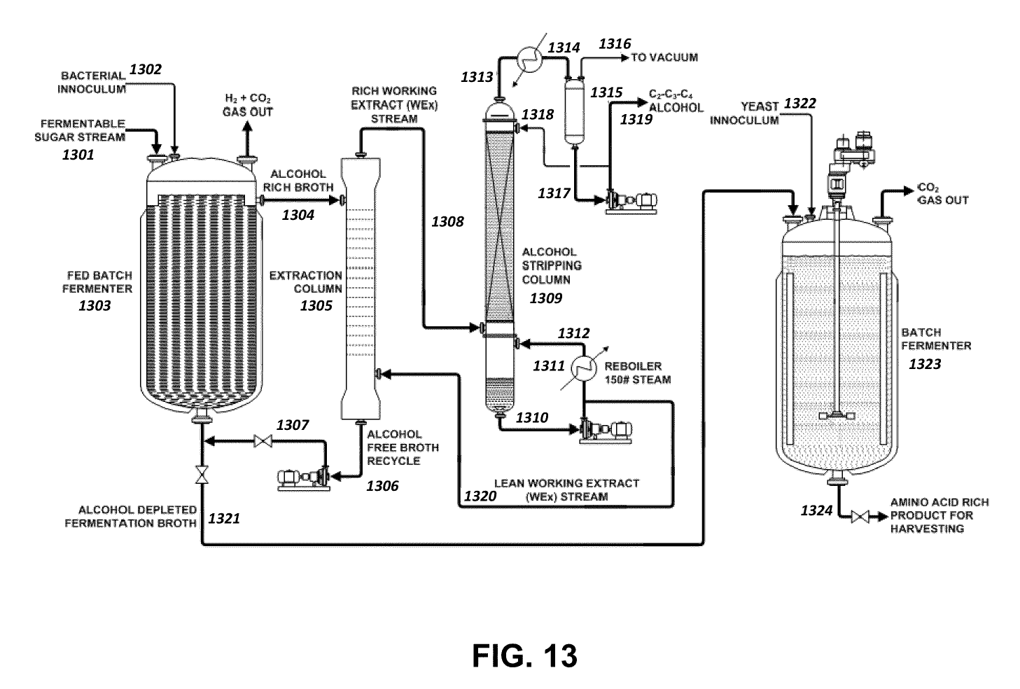

FIG. 13 illustrates another example to inhibit substrate inhibition of cells. Fermentable sugar stream 1301 is introduced into the fermenter using a fed-batch operation. This technique is used to inhibit substrate inhibition of the cells by high concentrations of sugar in the fermenter. The inoculum 1302 is introduced into the fermenter during the initial fill-up stage. Fermentation is conducted in the fermenter 1303 and yields a broth that includes the C₂—C₃—C₄ alcohol product, comprising primarily the C₄, or butanol fraction.

Broth 1304 is circulated from the fermenter to extraction column 1305. The extraction column is a multi-stage column that works in countercurrent mode. The broth flows down through the column. The extractant flows up through the column. The multi-stage configuration allows the extraction to be performed efficiently. Each stage may contain packing; or, more preferably, may have include tray with a large number of holes punched into the tray as a sieve. Sieve trays are efficient devices for countercurrent operation where the dense phase, in this case the broth, flows down and the light phase, in this case the oil extractant, flows up. Intense mixing occurs between these two phases as pass through the small holes in the trays. In the countercurrent extraction column the inhibitory fermentation products such as butanol, propanol and ethanol are transferred into the lean working extract. The alcohol-free broth 1306 is pumped back to the fed batch fermenter. This stream 1307 is sparged into the bottom of the fermenter to allow upward flow of the broth within the fermenter. The rich working extract 1308, containing the C₂—C₃—C₄ alcohols, is directed into the alcohol stripping column 1309 where the alcohols are distilled out as the C₂—C₃—C₄ alcohol product.

The alcohol stripping column is designed to take the bottoms 1310 from the column and pump them through a low-pressure steam reboiler 1311 with sufficient vacuum to strip the alcohols out. The exiting liquid from the reboiler 1312 is recirculated back into the bottom of the column. The stripped vapors from the top of the column 1313, comprising primarily the C₂—C₃—C₄ alcohols, flow through a condenser, and the condensed liquid 1314 is collected in an overhead receiver 1315. Appropriate recycles are maintained to ensure product purity. Vacuum 1316 is maintained to ensure appropriate vapor velocities within the stripping column. Liquids from the overhead receiver 1317 flow into a pump, where a portion of the pumped stream 1318 is refluxed back into the column. The balance is pumped out as C₂—C₃—C₄ alcohol product 1319. The overhead alcohol product (C₂—C₃—C₄ alcohol) is pumped to storage tanks.

The lean working extract stream 1320 that is produced at the bottom of the alcohol stripping column is recycled back into the bottom of the extraction column.

Butanol and isopropanol are maintained below their inhibitory levels in the fed-batch fermenter, where fermentation is carried out for about 52 hours. The flow of sugar into the fermenter is stopped near the end of the fermentation cycle to allow some residual sugar to be consumed. After the cycle is completed, the alcohol-depleted fermentation broth 1321 is withdrawn from the fermenter. This stream is blended with a yeast inoculum 1322 and pumped into another batch fermenter 1323. An appropriate fermentation cycle is conducted in this batch fermenter and the residual sugars and alcohol in the depleted fermentation broth are converted into an amino acid rich product 1324 for harvesting.

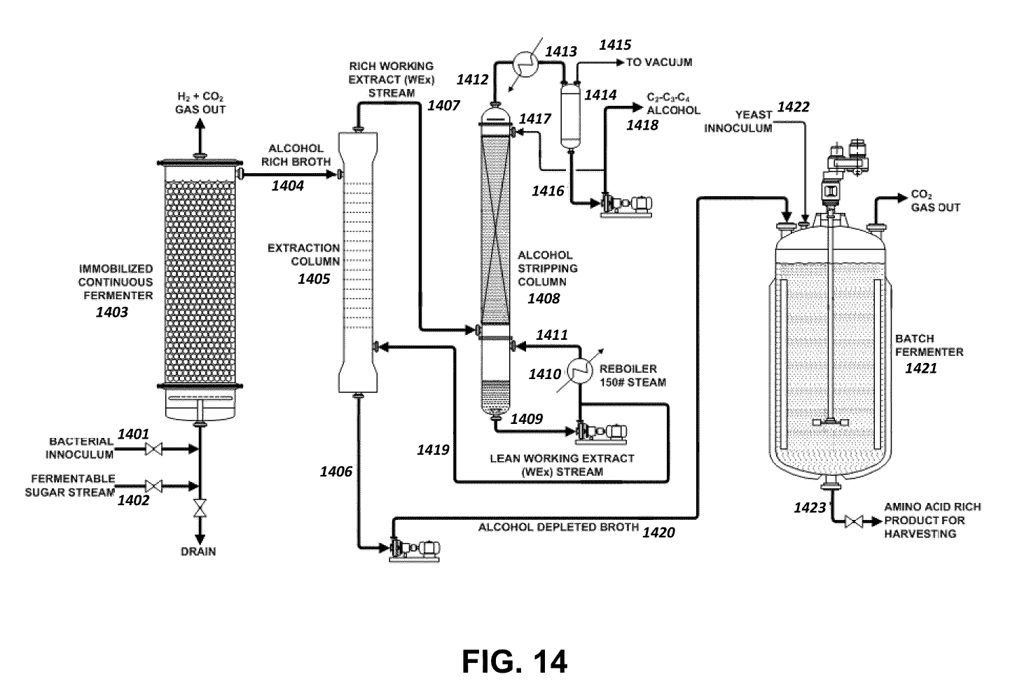

FIG. 14 illustrates another example utilizing immobilized continuous fermenters. Immobilized continuous fermenters are becoming more prevalent because of their ease of operation and the relative simplicity of maintaining sterile conditions. In these fermenters, the organism is typically maintained and grown on a porous substrate. Examples of porous substrate include porous glass beads, sintered metal beads, and extruded silica and silica-alumina structures that can be maintained in a fixed bed configuration.

Bacterial inoculum 1401 is introduced from the bottom of immobilized bed fermenter 1403 for a time to sufficient for it to set appropriately on the porous substrate. After the introduction phase, the fermentable sugar stream 1402 is introduced into the bottom of the fermenter through a sparger ring. Once the fermentable sugar stream starts flowing into the immobilized bed fermenter, the process operates in a continuous mode. Unless there is an “upset” condition or need for periodic maintenance, the operation will run continuously for a prolonged period of time. Hydrogen and CO₂ gases produced during the fermentation process can be readily vented, preferably from the top of the immobilized bed fermenter.

Fermentation is conducted in the fermenter and yields a broth 1404 that includes the C₂—C₃—C₄ alcohol product comprising primarily the C₄, or butanol fraction. The broth overflows from the top of the fermenter 1403 into extraction column 1405. The extraction column is a multi-staged column that works in a countercurrent mode of operation. The broth flows down through the column and the extractant, typically a vegetable oil or vegetable oil-containing oil mixture, flows up through the column. The multi-stage configuration allows the extraction to be highly efficient. Each stage may include packing, or more preferably a tray with a large number of holes to act as a sieve. Sieve trays are efficient for countercurrent operations where the denser phase, in this case the broth, flows down and the lighter phase, in this case the oil extractant, flows up. Mixing occurs between the two phases as they traverse the small holes in the trays. In the countercurrent extraction column the inhibitory fermentation products, such as butanol, propanol, and ethanol, are transferred into the lean working extract. The alcohol-depleted broth 1406 is pumped from the liquid-liquid extraction column for re-use in a subsequent downstream operation. The rich working extract 1407, containing the C₂—C₃—C₄ alcohols, is directed to alcohol stripping column 1408, where the alcohols are distilled out as the C₂—C₃—C₄ alcohol product. Alternatively, the rich working extract 1407 may be directed into a holding tank to allow for phase separation, decanting of the rich working extract as a means to remove residual water, followed by the alcohol stripping column 1408.

The alcohol stripping column takes the bottoms 1409 from the column and pumps them through a low pressure steam reboiler 1410 under sufficient vacuum to strip the alcohols out. The liquid 1411 exiting the reboiler is recirculated back into the bottom of the column. The stripped vapors from the top of the column 1412, comprising primarily C₂—C₃—C₄ alcohols flow through a condenser. The condensed liquid 1413 is collected in an overhead receiver 1414, and appropriate recycles are maintained to ensure product purity. Vacuum 1415 is maintained to ensure appropriate vapor velocities within the stripping column. Liquids from overhead receiver 1416 flow into a pump. Part of the pumped stream 1417 is refluxed back into the column, and the balance is pumped out as C₂—C₃—C₄ alcohol product 1418. The overhead alcohol product (C₂—C₃—C₄ alcohol) is pumped to storage tanks The lean working extract stream 1419 that is produced at the bottom of the alcohol stripping column is recycled back into the bottom of the extraction column.

The alcohol-depleted broth 1420 is pumped into batch fermenter 1421 where the residual products of fermentation and extraction are used as the feed source to make an additional high value by-product. An example would be, where a yeast inoculum 1422 is introduced. An amino acid rich product 1423 is withdrawn from this fermenter upon completion of cycle. Alternatively, the alcohol depleted broth 1420 may be directed through a clarifying centrifuge, the remaining water and residual fermentation products recycled into the incoming sugar stream 1402. In addition, in the embodiment that uses the residual fermentation and extraction products to make an additional high value product, the residual water from that fermentation process can be directed through a clarifying centrifuge, the remaining water then directed to the sugar inlet stream 1402.

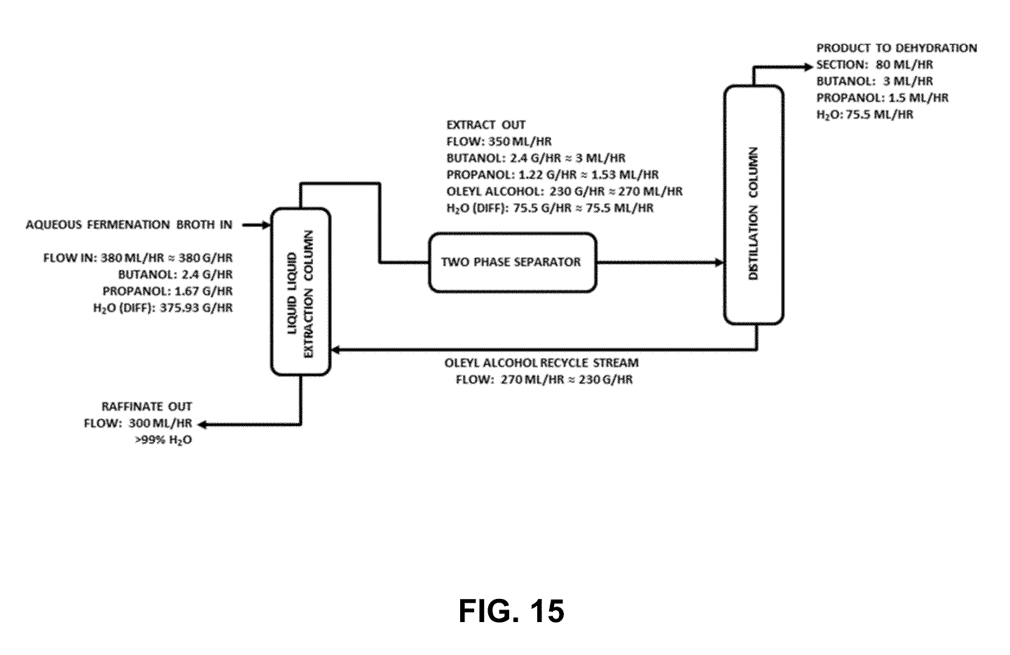

FIG. 15 illustrates an example of the extraction of alcohols in the fermentation broth using oleyl alcohol. Oleyl alcohol offers certain advantages over other substances. For example, can achieve very high single pass extraction of butenol. At the flow rates and mixing vigorousness desired does not emulsify to the same level other extractants do.

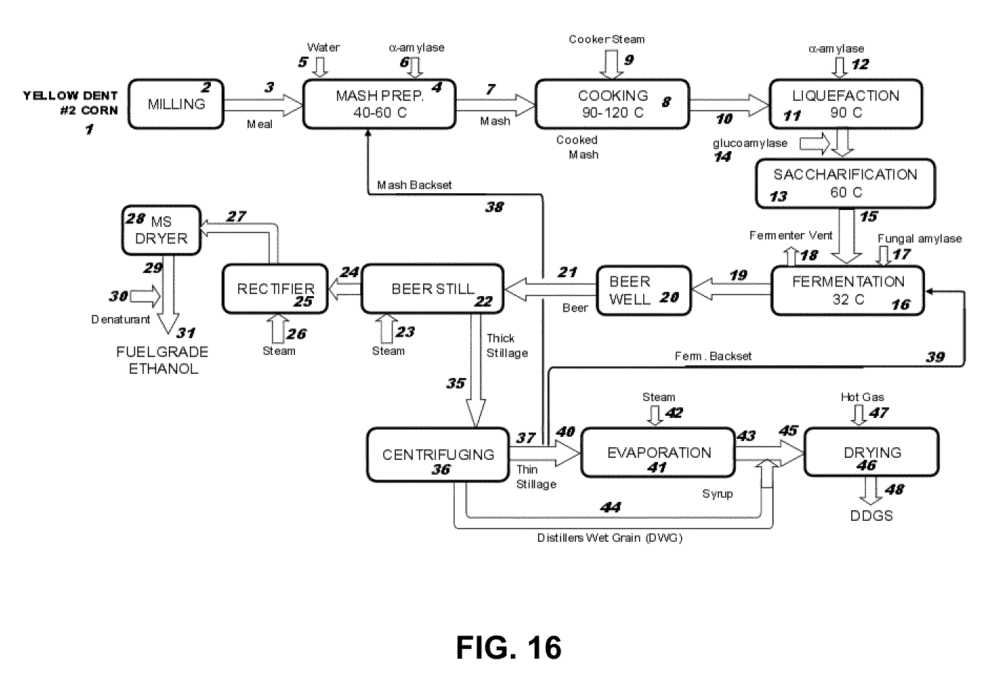

FIG. 16 illustrates a “dry mill” ethanol production facility.

The conventional “dry mill” process of producing fuel grade ethanol from corn proceeds through a sequence of unit operations.

Corn (maize) 1 first passes into milling area 2, where a hammer mill 3 breaks the corn into small particles to pass through #12 screens. The fineness of grind can be a significant factor in the final ethanol yield, so care should be taken to ensure quality.

The milled corn flows into the cooking section, which conducts mash preparation, cooking, liquefaction, and saccharification.

The foundation of cooking is 5-fold:

Sterilization. The mash should be sterilized to minimize levels of unwanted microorganisms.

Release of all bound sugars and dextrins (chains of sugars). Extraction allows subsequent enzymatic hydrolysis to use all available sugars. Starches (precursors of simple sugars), which are often bound to protein and fiber, are freed during cooking.

Protein breakdown to amino acids should be minimized. Amino acids and small peptides can bind sugars in Maillard reactions, which leaves sugar unavailable.

Solubilization of sugars. Sugars are solubilized, but only partially—typically 2-3% free sugars. Yeast growth can then occur rapidly, but not excessively as could happen if too much sugar were released at once.

Reduction in viscosity. Following gelatinization, viscosity is reduced to allow the slurry to more easily be moved through lines for subsequent processing.

“Cooking” is considered as the entire process, beginning with mixing the grain meal with water (and possibly also backset stillage), through the delivery of a mash that is ready for fermentation.

Mash preparation 4: allows for pre-liquefaction of the starch. In order for the α-amylase to gain access to the starch molecules, the granular structure of the starch must first be broken down in a process known as gelatinization. Gelatinization occurs to a greater or lesser degree during the mash preparation stage, where water 5 and α-amylase 6 are introduced to promote the reaction. The converted mash 7 flows into the next section.

Cooking 8: 150 psig steam 9 is injected into the mash, and the process of cooking is initiated. When the slurry of meal and water is cooked, the starch granules absorb water and swell. They gradually lose their crystalline structure and become large; gel-filled sacs that tend to fill available space, and also to break with agitation and abrasion. The cooked product 10 flows into the next section.

Liquefaction 11: this process enables α-amylase 12 to partially hydrolyze the exposed starch molecules into malto-dextrins. Starch commonly exists in two forms. One form is straight-chain amylose, in which the glucose units are bound by α-1,4 glucosidic linkages. The amylose content of corn is about 10% of the total starch; and the amylase chain length can be up to about 1,000 glucose units.

The other form of starch is amylopectin, which represents about 90% of the starch in corn. Amylopectin has a branched structure. It has the same α-1,4 glucosidic linkages as in amylose, but also has branches connected by α-1,6 linkages. The number of glucose units in amylopectin can be as high as 10,000. Corn, wheat, and sorghum (milo), the three most common feedstocks for ethanol production, have similar levels of starch, but the relative proportions of amylose and amylopectin differ with grain and even with varieties of the same type of grain.

The α-amylase enzyme acts randomly on the α-1,4 glucosidic linkages in amylose and amylopectin; but it will not break the α-1,6 linkages of amylopectin. The resulting shorter, straight-chain oligosaccharides are called “dextrins”; while the shorter, branched-chains are called “α-limit dextrins.” The mixture of dextrins is much less viscous than the starting material.

Saccharification 13 is the release of individual glucose molecules from the liquefied mixture of dextrins of varying sizes. The exoenzyme glucoamylase 14 releases single glucose molecules by hydrolyzing successive α-1,4 linkages, beginning at the non-reducing end of the dextrin chain. Glucoamylase also hydrolyzes α-1,6 branch linkages. The saccharified stream 15 flows into the next section.

Fermentation 16: The yeast amylase 17 converts sugar to ethanol. The process of converting sugars to ethanol takes between 10 and 60 hrs, and generates heat.

The majority of the cooled mash flows to one of a battery of six fermenters. A small portion is instead transferred to the yeast slurry tank, where it is combined with a mixture of active dry yeast and enzymes. This mixture is held for approximately 10 hours. The hydrated and actively growing yeast, along with saccharifying enzymes, nutrients, and industrial antibiotics are added to the fermenter during filling.

The fermenter pump circulates the contents of the fermenter through the fermenter cooler. This prevents grain solids from settling and removes heat generated by fermentation. Carbon dioxide gas 18 generated during fermentation is vented to the beer well and then into an adsorber, before being removed from the system. Once the fermentation is complete, the product 19 is pumped into the next stage.

Fermenters typically have a cylindro-conical configuration with a skirt support. They are usually designed with a capacity from about 100,000 to about 500,000 gallons. A cooling system can use, for example, external cooling jackets. An agitator is typically used, especially at the start and at the end of the fermentation. Stainless steel is an example of a good material for this use, as it is easy to clean and to sterilize.

The fermenter is preferably cleaned with clean-in-place (CIP) equipment. The CIP system assists in controlling microbial contamination and consequent reduction in ethanol yield. A typical cleaning procedure is described below.

Initial rinse water is pumped to the equipment being cleaned with the CIP pump. This procedure takes about 10 minutes. Following this, detergent solution is circulated for about 20 minutes. The detergent can be based on caustic soda, normally with added wetting agent, antifoamer, and descaling agent. The caustic strength should normally be in the 3-5% range. The detergent should be hot (80-90° C.). A post rinse is conducted for about 10 minutes to eliminate the residual caustic from the equipment and piping.

Beer well 20: when fermentation is complete, the beer is transferred to the beer well via the fermenter pumps. The beer well also provides surge capacity between fermentation and distillation.

The beer stream 21, which contains approximately 10.0% w/w ethanol, is preheated from the normal fermentation temperature in several stages, recovering low level and intermediate level heat from effluent streams and vapors in the process. This preheated beer is degassed and fed to the beer still 22, which has stripping trays below the beer feed point and several rectifying trays above. Steam 23 is injected into the still to facilitate stripping. The condensed high wines 24 from the top of the still are fed to rectifying tower 25, which has an integral stripping section. Steam 26 is also introduced into the bottom of the tower to facilitate stripping. The high grade ethanol product 27, whether industrial or potable, is taken as a side draw from one of the upper trays. A small heads cut is removed from the overhead condensate. Fusel oils (mixtures of higher molecular weight alcohols such as propyl, butyl, and amyl alcohols and their isomers, or ‘congeners’) are drawn off at two points above the feed tray but below the product draw tray to avoid a buildup of fusel oil impurities in the rectifying tower. The overhead heads cut and the fusel oil draws are also sent to the concentrating tower.

The rectifying tower is heated by vapors from both the pressurized extractive distillation tower and the pressurized concentrating tower. In the concentrating tower, the various streams of congener-containing draws are concentrated. A small heads draw is taken from the overhead condensate, which contains the acetaldehyde fraction along with a small amount of the ethanol produced. This may be sold as a by-product or burned as fuel. A fusel oil side draw is taken at high fusel oil concentrations through a cooler to a washer. In the washer, water is used to separate ethanol from fusel oil, with the washings being recycled to the concentrating tower. The decanted fusel oil may be sold as a by-product.

In tray towers, vapor/liquid contact occurs on the individual trays by purposely interrupting down-flowing liquid using downcomers to conduct vapor-disengaged liquid from tray to tray, and causing the vapor/liquid contact to occur between cross-flowing liquid on the tray and vapor flowing up through the tray. The most commonly used type of tray is a sieve tray, one in which a large number of regularly oriented and spaced small circular openings have been drilled or punched.

Molecular sieve drying 28: the superheated ethanol vapor flows to molecular sieve units for dehydration. The vapor passes down through one bed of molecular sieve beads under pressure control. Ethanol vapor at a minimum concentration of 99.3% w/w ethanol exits the molecular sieve units. The molecular sieve units are cycled so that some are regenerating while others are adsorbing water from the hydrous ethanol vapor stream. The hot anhydrous ethanol vapor is condensed in condenser 29.

Fuel grade product blending: During transfer to final storage, the product is denatured by adding unleaded gasoline 30. The finished product 31 is transferred to tanks for loadout.

Centrifugation and Drying: thick stillage 35 from the bottom of the beer still is pumped to stillage centrifuges 36 that split the feed into two fractions: distillers wet grain 44 and thin stillage 37 and the concentrate. The wet grain contains approximately 33-35% w/w solids. The centrifuge is positioned to discharge the cake to a conveyor that feeds to the DDGS drying system.

The thin stillage contains approximately 6-8% w/w total solids, of which the majority are dissolved solids. A portion of the thin stillage 38 or “mashed backset” is used for final dilution and pH adjustment of the liquefied mash. Another portion 39 “fermentation backset” is used in the fermentation process. The balance of the thin stillage 40 is directed to the evaporation step 41. Steam 42 is used to provide the evaporative heat.

The distillers wet grain from centrifugation, and syrup from the evaporation area 43 are mixed in a mingler prior to the dryer inlet. The mixture 45 is conveyed into a rotary direct gas-fired dryer 46. Hot gases 47 are introduced into the dryer, where the cake solids concentration increases to approximately 90% w/w. The Distiller's Dried Grains with Solubles (DDGS) 48 product is conveyed to storage.

Evaporation: the evaporation step consists of a multi-effect evaporation system operating under vacuum on a continuous basis. The evaporator system removes water from the thin stillage, concentrating the total solids fraction to approximately 35% w/w solids. This stream is then sent to the DDGS dryer to be dried along with the cake to a solids content of 90% w/w. The evaporator condensate is recycled back to the process as dilution water for the mashing process and CIP rinses.

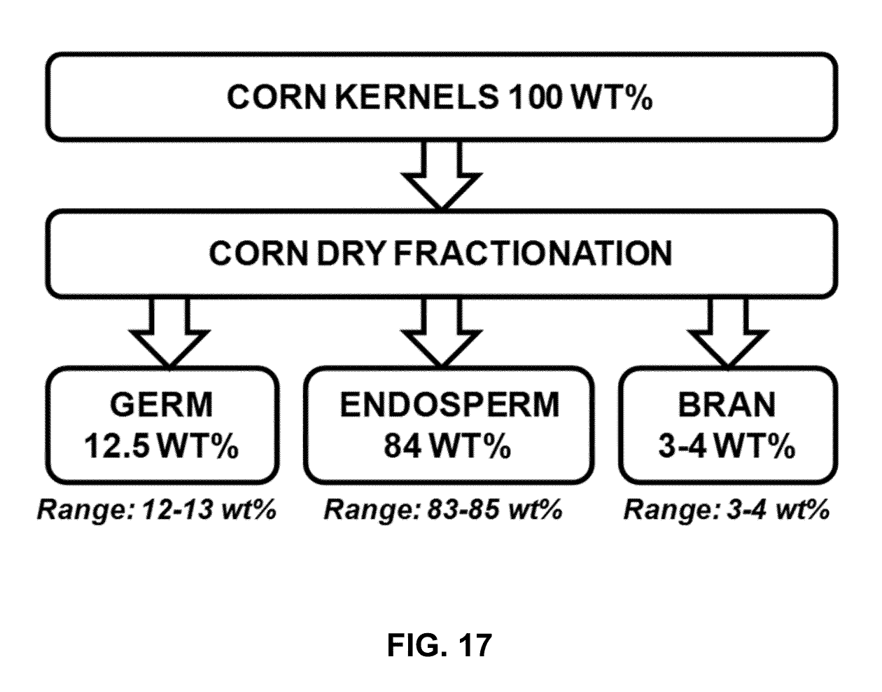

FIG. 17 illustrates the process of dry fractionation. The feed for the converted Optinol facility is corn endosperm from dry fractionation. “Dry fractionation” is also known as “dry milling.” The corn kernel is separated into three main components: corn starch, germ, and bran. The starch is sent to the fermenter and the germ is sent to an oil extraction facility.

The germ is the living portion of the corn kernel. It contains the genetic information, enzymes, vitamins and minerals for the kernel to grow into a corn plant. About 25% of the germ is corn oil. Corn oil is the most valuable part of the corn kernel due to the amount of linoleic fatty acid (polyunsaturated fat) it contains, and its bland taste. The corn germ contains about 85% of the total oil of the kernel.

The endosperm holds about 82% of the kernel's dry weight. It is the source of energy, protein, and starch for the germinating seed. There are two types of endosperm, soft and hard. In a hard endosperm, starch is tightly packed. In a soft endosperm, the starch is more loose. When corn dries in the field before harvest, the soft endosperm collapses as it dries, and forms a characteristic dent in the top of the kernel.

The “bran fraction” contains the pericarp, the outer covering of the kernel. It resists water and water vapor and protects against insects and microorganisms. The bran fraction also includes the tip cap, the only area of the kernel not covered by the pericarp, the former attachment point of the kernel to the cob. The tip cap is the major entry path into the kernel.

A typical analysis of dry fractionation product composition is shown in Table 16.

TABLE 16
Analysis of dry fractionation product composition

| Corn | | Protein, | | | |
|---|---|---|---|---|---|
| Component | Starch, wt % | wt % | Oil, wt % | Fiber, wt % | % Total |
| Endosperm | 82.6 | 11.7 | 1.8 | 3.9 | 83.6 |
| Germ | 24.8 | 36.5 | 22.2 | 16.5 | 11.2 |
| Bran | 18.0 | 54.9 | 6.3 | 20.8 | 5.2 |

Adding fractionation to an alcohol-production process splits the corn kernel into its main components at the outset. This approach can increase the efficiency of fermentation by concentrating the starch, and diverting the proteins, oils, and fiber to other uses. The oil may be used directly in the same plant, for example, as the extractant oil as previously described.

Incoming corn is separated into its main components by means known in the art, typically either by mechanical or abrasion milling. A degerminator forces the entire kernel against a screen, pushing the germ portion through while retaining the endosperm. The endosperm and bran are then further separated, and these three streams are available for their respective, separate processing.

Isolating the starch—the principal fermentable component of the corn kernel—boosts the efficiency of the ethanol production process. With less non-fermentable material going through the process, energy consumption is reduced. There can be a 15 to 20 percent reduction in material to dry at the back end of the process, which leads to significant energy savings. Less drying may also lead to lower emissions from the plant.

In addition, the higher concentration of starch in the stream leads to more efficient fermentation and distillation. There should be a lower demand for enzymes, and less energy needed to separate ethanol from the beer. The cleaner starch stream may experience less fouling, and reduced chemical usage for cleaning.

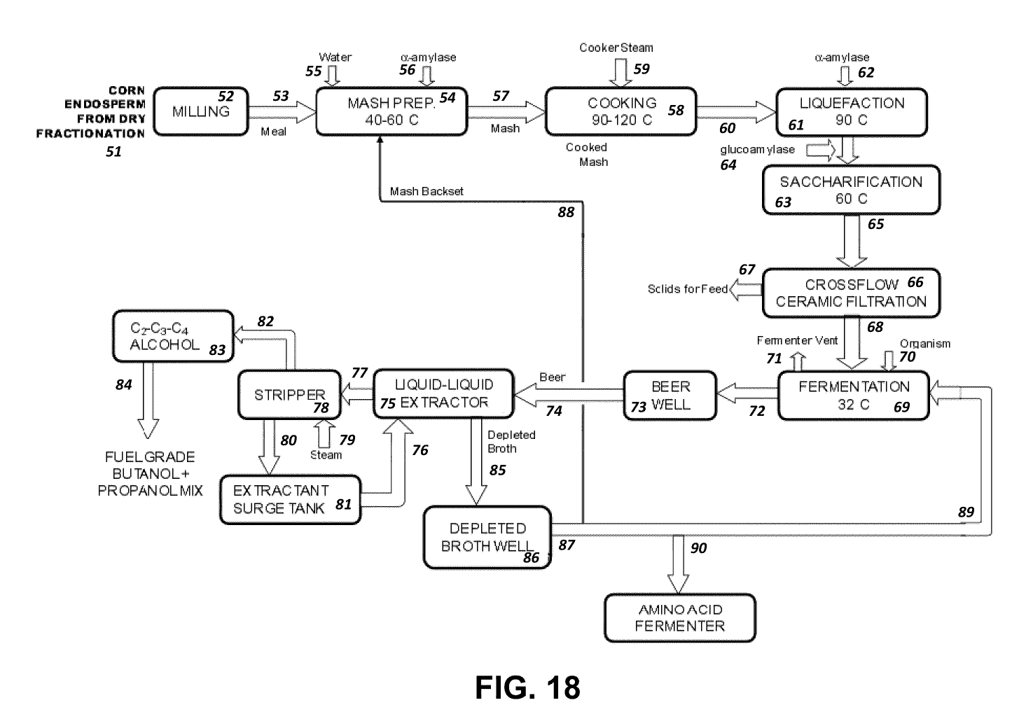

FIG. 18 illustrates an example in which a “dry mill” ethanol production facility such as that illustrated in FIG. 16 is converted into an Optinol production facility.

Operations from 51-65 can remain essentially unchanged from operations 1-15 described above with respect to FIG. 16 for the traditional “dry milling” process for making ethanol from corn. Endosperm contains about 80-85% starch. To the extent feasible, it can help improve process efficiency to separate the other components of the endosperm from the saccharified stream. These other components comprise primarily protein and fiber. The saccharified stream is directed through a filtration system 66 which separates the solids 67 from the saccharified stream. Any filtration methodology may be conveniently used as practiced in the art. Filtration methodologies include, for example, centrifuging, frame filtration, rotary drum filtration, and other means known in the art. An example of a specific method is to use cross-flow ceramic filters. The solids stream is mostly a sludge, and may be handled accordingly. It may be dried or dewatered, and the water stream may be directed back into the mash preparation section.

The stream leaving the filtration operation 68 is pumped into the pre-existing fermentation vessel 69. The organism 70 introduced into the fermenter is the novel organism discussed earlier. This organism is specifically to convert the sugar in the feed stream into C₂—C₃—C₄ alcohol product. Fermentation is conducted on a fed batch basis and the gases emanating from the fermentation cycle 71 comprise of an equimolar stream of H₂ and CO₂. This stream may be sent to the facilities boiler or in a subsequent downstream retrofit may be used to produce methanol on site. Once the fermentation cycle has been successfully concluded, the C₂—C₃—C₄ alcohol rich beer is dropped into the pre-existing beer well 73.

The beer from well 73 is pumped into the overhead section of liquid-liquid extractor 75. The liquid-liquid extractor is, for example, a modification of the pre-existing beer still. Conventional beer stills use sieve trays within the stills to manage the effective number of mass transfer stages. In the modification of the conventional beer still, the sieve trays are retained and reconfigured to function as liquid-liquid extraction trays, in which the heavy phase, comprising stream 74, flows down through the sieve holes. The extractant stream 76, comprising a mixture of triglycerides and mineral oil, is directed into the bottom of the liquid-liquid extractor and flows upwards due to its being less dense. Intense mixing between the downflow heavy phase and the upflow light phase occurs within each stage in the perforated trays. The C₂—C₃—C₄ alcohols are extracted into the light oil extractant phase and leave the extractor as stream 77.

This extractant stream containing C₂—C₃—C₄ alcohols enters the top of the stripper 78. The stripper 78 is a modified pre-existing rectifier. The hydrodynamics within the column are modified to accept a downflow of extractant oils (rather than the heavier water phase). Steam 79 is injected into the stripper through a pre-existing nozzle. The steam supply is also typically a pre-existing facility of the plant. The lean extractant that flows down the stripper column exits as stream 80 and is directed into surge tank 81. This lean extractant solution is pumped back into the liquid-liquid extractor as stream 76. The extractant surge tank 81 will typically need to be added when one modifies a pre-existing ethanol production facility.

The high grade C₂—C₃—C₄ alcohol product is stripped out as stream 82 and is condensed and directed into overhead receiver 83. Fuel grade C₂—C₃—C₄ alcohol mix 84 is pumped out through pre-existing load out facilities.

The depleted broth stream 85 from the liquid-liquid extractor is directed into depleted broth well 86. This depleted broth well may need to be added if an existing tank is not already available. Stream 87 is continuously pumped from this well and split into three major streams. One stream 88 is directed into the mash preparation section 54 as the mash backset stream. Another stream 89 is directed back into fermenter section 69. The majority of the depleted broth stream 90, still containing a low level of unextracted C₂—C₃—C₄ alcohol and a low level of unconverted sugars, is directed to a new fermenter (which could be a pre-existing ethanol fermenters), to convert the residual alcohol and sugars into amino acids. The amino acids can be sold as a commercial product.

The complete disclosures of all references cited throughout this specification are hereby incorporated by reference. In the event of an otherwise irreconcilable conflict, however, the present specification shall control. This incorporation by reference includes, where applicable, the complete disclosures of the supplementary information made available by the publishers on the Internet.

What is claimed is:

1. A method comprising:
(a) fermenting a suitable feedstock with Clostridium under conditions amenable to the production of butanol;
(b) mixing a non-toxic oil with the fermentation mixture, to produce an oil-water foam on the surface of the mixture, wherein the butanol preferentially partitions into the foam;
(c) separating at least part of the foam from the fermentation mixture;
(d) resolving the foam into an oil phase, an alcohol phase, and an aqueous phase;
(e) separating the alcohol phase and extracting butanol from the alcohol phase.

2. The method of claim 1, wherein the suitable feedstock is selected from the group consisting of sugarcane juice, sugarcane molasses, corn, sweet sorghum juice, sugar beet molasses, cheese whey, corn steep liquor, and combinations of these.

3. The method of claim 1, further comprising fermenting the suitable feedstock with Clostridium.

4. The method of claim 1, further comprising fermenting the suitable feedstock with a preferred species and strain as per American Type Culture Collection (ATCC) deposit.

5. The method of claim 1, further comprising fermenting a suitable feedstock with Clostridium under conditions amenable to the production of butanol without substantial acetone.

6. The method of claim 1, further comprising fermenting a suitable feedstock with Clostridium under conditions amenable to the production of butanol and isopropanol.

7. The method of claim 6, further comprising mixing a non-toxic oil with the fermentation mixture, to produce an oil-water foam on the surface of the mixture, wherein the butanol and isopropanol preferentially partitions into the foam.

8. The method of claim 6, further comprising separating the alcohol phase and extracting at least a portion of the butanol and isopropanol from the alcohol phase.

9. The method of claim 6 comprising separating the alcohol phase and extracting at least a portion of the butanol and isopropanol from the alcohol phase by distillation.

10. The method of claim 1, further comprising fermenting a suitable feedstock with Clostridium under conditions amenable to the production of butanol and isopropanol without substantial acetone.

11. The method of claim 1, wherein the non-toxic oil is selected from the group consisting of corn oil, canola, vegetable oil, mineral oil, oleyl alcohol, and combinations of these.

12. The method of claim 1, further comprising resolving the foam using antifoaming agent.

13. The method of claim 1, further comprising resolving the foam using salt.

14. The method of claim 1, further comprising resolving the foam using mechanical means.

15. The method of claim 1 comprising separating the alcohol phase and extracting at least a portion of the butanol from the alcohol phase by distillation.

16. The method of claim 1, further comprising returning the oil phase to the fermentation mixture.

17. The method of claim 1, further comprising purifying the oil.

18. The method of claim 1, further comprising performing countercurrent extraction.

19. The method of claim 1, further comprising performing steps (a) to (e) as a continuous process.

20. A Clostridium bacteria of strain Clostridium beijerinckii sp. optinoii, wherein a representative sample of Clostridium beijerinckii optinoii has been deposited under American Type Culture Collection (ATCC) accession number PTA-11285.
